(19) United States
(12) Patent Application Publication
(10) Pub. No.: US 2023/0081539 A1
(43) Pub. Date: Mar. 16, 2023

(54) SUPRACHOROIDAL DELIVERY OF DRUG PARTICLES TO REDUCE TOXICITY

(71) Applicant: The Johns Hopkins University, Baltimore, MD (US)

(72) Inventors: Jie Fu, Baltimore, MD (US); Justin Hanes, Baltimore, MD (US); Peter Campochiaro, Baltimore, MD (US); Laura Ensign, Baltimore, MD (US)

(21) Appl. No.: 17/800,838

(22) PCT Filed: Feb. 19, 2021
(86) PCT No.: PCT/US2021/018767

§ 371 Date: Aug. 18, 2022

Related U.S. Application Data
(60) Provisional application No. 62/980,055, filed on Feb. 21, 2020.

(51) Int. Cl. A61K 31/765; A61K 31/704; A61K 31/7084; A61K 9/00; A61K 9/10

(52) U.S. Cl. CPC A61K 31/765; A61K 31/704; A61K 31/7084; A61K 9/0048; A61K 9/10

(57) ABSTRACT

A population of polymeric particles for controlled release of therapeutic agents which have unacceptable toxicity when administered intravitreally can be safely administered suprachoroidally at the same intravitreal concentration or dose. In a preferred embodiment, the particles have a high loading of the agent and is released without a substantial initial burst release. Examples demonstrate safety and efficacy of delivery of acriflavine-containing particles when administered suprachoroidally. The examples demonstrate sustained release with low to no burst release of the highly water soluble agent for up to 60 days.

### CROSS-REFERENCE TO RELATED APPLICATIONS

This application claims benefit of U.S. Provisional Application No. 62/980,055, filed on Feb. 21, 2020, which is hereby incorporated herein by reference in its entirety.

### STATEMENT REGARDING FEDERALLY SPONSORED RESEARCH

This invention was made with Government support under Grant No. EB016121 awarded by the National Institutes of Health. The Government has certain rights in the invention.

### FIELD OF THE INVENTION

The invention is generally directed to the field of delivery of therapeutic agents to the eye, where the agent is formulated in particles for controlled release and administered suprachoroidally to significantly decrease toxicity.

### BACKGROUND OF THE INVENTION

Therapeutic, prophylactic and/or diagnostic agents may be delivered locally, often in controlled delivery systems. The controlled delivery systems provide release of the agent at a specific site for defined period of time(s). There are therapeutic benefits to providing an active agent to a specific target tissue, instead of systemically. This is because the effect of the agent on the target tissue can be maximized while the side effects on other tissues can be minimized. Providing controlled release of the agent further enhances benefit. Controlled release of an agent allows the concentration of the agent at the target tissue site to remain at a more consistent therapeutic level, over a period of time. This reduces the number of administrations of the agent to the local site, as well as avoids the peaks and troughs of drug concentration found with traditional drug therapies.

Some types of agents, however, may be difficult to regulate when provided in the form of an agent-eluting controlled delivery system. It is difficult to capture therapeutic agents in most controlled delivery systems due to difficulties in regulating the elution rate of the therapeutic agents from controlled delivery systems. High initial burst release of therapeutic agents from controlled delivery systems is one of the major challenges. Too high a burst reduces the effective lifetime of the drug delivery device, reducing its effectiveness both therapeutically and economically. Even worse, excessive initial release rates can result in drug levels close to or exceeding toxic threshold levels.

The eye is a particularly difficult delivery site due to multiple compartments, with different pharmacokinetics, and the risk of toxicity associated with release of an agent within a space that does not freely diffuse out. This is complicated by the desire to have as few injections as possible, to minimize pain and inconvenience, as well as costs, to the patient. While intravitreal injections are currently the mainstay of treatment for a wide variety of retinal disorders, some drugs can cause ocular complications, including elevated pressure, abnormal electroretinograms, and abnormal histology.

Topical administration of drugs is not commonly used for the treatment of retinal diseases because the limited attempts in which they have been tried have not shown success. Systemic treatment is not usually effective because the blood-retina barrier limits the ability of many drugs to reach the eye, and high doses can cause systemic adverse effects.

Therefore, it is the object of the present invention to provide controlled delivery systems for sustained local delivery of therapeutic agents over time.

It is another object of the present invention to provide methods of safely administering the controlled release systems into the eye, with minimal toxicity.

### SUMMARY OF THE INVENTION

A population of polymeric particles for controlled release of therapeutic agents which have unacceptable toxicity when administered intravitreally can be safely administered suprachoroidally at the same intravitreal concentration or dose. In a preferred embodiment, the particles have a high loading of the agent and is released without a substantial initial burst release. As used herein, the absence of a substantial initial burst release refers to a burst release of less than 50% of the agent within one day in an isotonic solution at 37° C., preferably less than 40, 30, 25, 20, 15, or 10%. The particles may include a therapeutic agent at a cumulative therapeutic or prophylactic dose between about 10 μg/mg and about 200 μg/mg particles. The therapeutic agent is typically in an amount between about 0.1% and 20% of the weight of the particle. The particles have an average diameter between about 100 nm and about 100 μm, preferably between about 1 μm and about 80 μm, more preferably between about 1 μm and about 60 μm, most preferably between about 1 μm and about 40 μm. Examples demonstrate efficacy and safety using acriflavine-containing particles.

The population of particles is administered suprachoroidally (i.e., in the suprachoroidal space), preferably using a trocar/cannula system. For example, the cannula portion is inserted obliquely through the conjunctiva and then the sclera. The trocar is slowly removed, leaving the tip of the cannula sitting in the subchoroidal space. A 30-gauge needle attached to a syringe containing fluid containing the particles is inserted through valves into the cannula and the contents are slowly injected into the suprachoroidal space. Other techniques such as microneedles, or a microneedle syringe which is suitable for suprachoroidal administration such as the CLEARSIDE® Microinjector can be used. The microinjedor is a syringe with a needle that is approximately 1 mm in length. This syringe utilizes a needle that is distinct from needles for administration via other routes of administration, such as intravitreal. The injector includes a 30-gauge needle, within a specially designed hub that allows the user to reliably inject the needle into the suprachoroidal space for drug administration.

The particles typically release the therapeutic agent at a steady rate over a period of time of at least two weeks, at least four weeks, at least six weeks, or at least eight weeks in vivo, or as measured in an isotonic aqueous solution at 37° C. The combination of no to minimal burst release, sustained release for a prolonged period of time and the route of administration reduces the toxicity associated with delivery of the therapeutic or even the same particles intravitreally, to levels acceptable to regulatory agencies such as the Food and Drug Administration.

Examples demonstrate safety and efficacy of delivery of acriflavine-containing particles when administered suprachoroidally. Hypoxia-inducible factor-1 (HIF-1) has been implicated in the pathogenesis of choroidal neovascularization (NV) and is a preferred target because it increases multiple pro-angiogenic proteins and their receptors. Acriflavine (ACF) binds HIF-1α and HIF-2α, thereby preventing binding to HIF-1β, thereby inhibiting transcriptional activity of HIF-1 and HIF-2. Delivery of ACF to the eye by multiple routes strongly, but transiently, suppresses choroidal NV. The examples demonstrate sustained release with low to no burst release of the highly water soluble ACF from poly(lactic-co-glycolic acid) (PLGA) microparticles (PLGA-ACF MPs in vitro for up to 60 days. Intravitreous injection of PLGA-ACF MPs in mice suppressed choroidal NV for at least 9 weeks and suprachoroidal injection of PLGA-ACF in rats suppressed choroidal NV for at least 18 weeks. Intravitreous, but not suprachoroidal injection, of PLGA-ACF MPs containing 38 μg of ACF in rabbits resulted in modest reduction of full-field electroretinogram (ERG) function. Over the span of 28 days after suprachoroidal injection of PLGA-ACF MP, rabbits had normal appearing retinas on fundus photographs, normal electroretinogram scotopic a- and b-wave amplitudes, no increase in intraocular pressure, and normal retinal histology. The active components of ACF, trypaflavine, had steady-state levels in the low nM range in RPE/choroid>retina for at least 16 weeks with a gradient from the side of the eye where the injection was done to the opposite side.

### BRIEF DESCRIPTION OF THE DRAWINGS

FIG. 1A is a graph showing ACF release in vitro (37° C., pH 7.4 PBS) shown as a percentage of the initial drug loading (mean±SEM, n=3) over time (days). FIG. 1B is a line graph showing the kinetics of acriflavine release (%) over time (days) from PLGA-ACF MPs prepared at different formulations (see Tables 1-14). Results in FIG. 1B is representative of results for all formulations tested. FIG. 1C is a line graph showing the kinetics of acriflavine release (%) over time (days) from ACF-PSA-PEG₃ MPs. Acriflavine microparticles (13.5 μm, loading 3.5%) in vitro (pH 7.4, 37° C.) release profile.

FIG. 2A is a diagram showing SCS delivery of PLGA microparticles (MPs) containing acriflavine (PLGA-ACF). SCS delivery of PLGA-ACF MPs delivers pharmaceutically relevant levels of ACF throughout the eye that significantly reduces the development of laser-induced choroidal neovascularization in rats. FIG. 2B is a bar graph showing the effect of acriflavine in a mouse CNV model. Mean area of CNV (mm²) in CNV mice treated with blank microparticles (blank, n=9) or microparticles with acriflavine (Acr, n=8) is shown. FIG. 2C is a bar graph showing the effect of acriflavine in the mouse ROP model. Mean area of NV (mm²) in ROP mice treated with blank microparticles (blank, n=7) or microparticles with acriflavine (Acr, n=7) is shown. FIG. 2D is a line graph showing the mean area of Choroidal Neovascularization (CNV) (mm²) in mice over time (weeks) after intravitreous injection of MP to CNV induction with treatments of PLGA only MPs (filled squares) or PLGA-ACF MPs (open squares). Results from an image analysis showed a significant reduction in mean (±SEM) area of CNV in eyes injected with PLGA-ACF MPs compared with those injected with empty PLGA MPs at 2, 4, and 8 weeks after injection. (** p<0.01, * p<0.05 by unpaired t-test).

FIG. 3 is a line graph showing the mean area of CNV (mm²) in rats over time (weeks) after injection of MP in the suprachoroidal space (SCS) to CNV induction with treatments of PLGA only MPs (filled squares) or PLGA-ACF MPs (open squares). Results from image analysis showed a significant reduction in mean (±SEM) area of CNV in eyes injected with PLGA-ACF MPs compared with those injected with empty PLGA MPs at all time points (** p<0.01, * p<0.05 by unpaired t-test).

FIG. 4 is a graph showing change in intra-ocular pressure (ΔIOP from baseline [mmHg]) over time (days) for rabbit eyes treated with: no injection; SCS-injected PLGA-ACF MPs with PVA coating, or PLGA MPs with PVA coating.

FIGS. 5A-5D are graphs showing the change in drug concentration (nM) over weeks after microparticle injection for trypaflavine (TRF) (filled circles) and proflavine (PRF) (filled squares) after measurement of the components of acriflavine in retinal pigmented epithelium (RPE)/choroid and retina following suprachoroidal injection of PLGA-ACF MPs. Each point represents the mean (±SEM).

FIG. 6 is a graph showing acriflavine suppression of CNV (Mean Area of CNV (mm²)) is due to the trypaflavine (TRF) component in the ACF, but not proflavine (PRF).

FIG. 7A is a graph showing change in IOP (ΔIOP from baseline [mmHg]) over time (days) for rabbit eyes treated with: no injection (1); SCS-injected PLGA-ACF MPs with PVA coating (2), or IVT-injected PLGA-ACF MPs with PVA coating (3). A shift in the ERG scotopic (FIG. 7B) a-wave and (FIG. 7C) b-wave was observed in the rabbits receiving IVT injections.

FIG. 8A is a graph showing change in IOP (ΔIOP from baseline [mmHg]) was elevated in rabbit eyes receiving IVT DXR-PSA-PEG₃ microparticles, leading to the sacrifice of animals on days 7 and 28. SCS injection of the same microparticles at the same dose did not significantly affect IOP. FIGS. 8B and 8C are electroretinogram data showing change in amplitude (μV) of scotopic a-wave and scotopic b-wave at different flash intensities (cd−s/m²) of treated eyes as compared to untreated eyes of rabbits with: untreated; or SCS-injected DXR-PSA-PEG₃ MPs (FIGS. 8B (a-wave) and 8C (b-wave)).

### DETAILED DESCRIPTION OF THE INVENTION

### I. Definitions

“Biodegradable Polymer,” as used herein, generally refers to a polymer that will degrade or erode by enzymatic action and/or hydrolysis under physiologic conditions to smaller units or chemical species that are capable of being metabolized, eliminated, or excreted by the subject, within a period of less than two years, more typically less than one year, although some will degrade in a period of hours, days, weeks or months. The degradation time is a function of polymer composition, morphology, such as porosity, particle dimensions, and environment.

“Hydrophilic,” as used herein, refers to the property of having affinity for water. For example, in the context of the agent, the therapeutic agent has a solubility between about 1 mg/ml and 500 mg/ml in room temperature water. In general, the more hydrophilic an agent is, the more that agent tends to dissolve in, mix with, or be wetted by water at room temperature and pressure.

An active agent is a substance that is administered to a patient for the treatment (e.g., therapeutic agent), prevention (e.g., prophylactic agent), or diagnosis (e.g., diagnostic agent) of a disease or disorder.

“Initial burst release” refers to the initial bolus of drug released upon placing the particles in the release medium. The release medium may be water, an isotonic solution, a physiological solution, or an in vivo environment. The release is measured at physiologically relevant temperatures, such as at about 37° C. For example, a “substantial initial burst release” refers to an initial burst release of greater than 50% of the agent within one day in an isotonic solution at 37° C.

As used herein the term “effective amount” or “therapeutically effective amount” means a dosage sufficient to treat, inhibit, or alleviate one or more symptoms of a disease or disorder being treated or to otherwise provide a desired pharmacologic and/or physiologic effect. The precise dosage will vary according to a variety of factors such as subject-dependent variables (e.g., age, immune system health, etc.), the disease or disorder, and the treatment being administered. The effect of the effective amount can be relative to a control. Such controls are known in the art and discussed herein, and can be, for example the condition of the subject prior to or in the absence of administration of the drug.

As used herein, the term “treating,” or “treat” refers to arresting or inhibiting, or attempting to arrest or inhibit, the development or progression of a disease and/or causing, or attempting to cause, the reduction, suppression, regression, or remission of a disease and/or a symptom thereof. As would be understood by those skilled in the art, various clinical and scientific methodologies and assays may be used to assess the development or progression of a disorder, and similarly, various clinical and scientific methodologies and assays may be used to assess the reduction, regression, or remission of an infection or its symptoms. “Treatment” refers to both therapeutic treatment and prophylactic or preventative measures.

“Nanoparticle,” as used herein, generally refers to a particle having an average diameter from about 10 nm up to but not including about 1 micron. The particles can have any shape. Nanoparticles having a spherical shape are generally referred to as “nanospheres”. “Microparticle,” as used herein, generally refers to a particle having a diameter, such as an average diameter, from about 1 micron to about 100 microns. The microparticles can have any shape. Microparticles having a spherical shape are generally referred to as “microspheres”.

“Molecular weight,” as used herein, generally refers to the relative average chain length of a polymer, unless otherwise specified. In practice, molecular weight can be estimated or characterized using various methods including gel permeation chromatography (GPC) or capillary viscometry. GPC molecular weights are reported as the weight-average molecular weight (Mw) as opposed to the number-average molecular weight (Mn). Capillary viscometry provides estimates of molecular weight as the inherent viscosity determined from a dilute polymer solution using a particular set of concentration, temperature, and solvent conditions.

“Mean particle size,” “average particle size,” and the following related terms when referring to a particle(s): “average size,” “mean size,” “average diameter,” and “mean diameter,” as used herein, generally refer to the statistical mean particle size (diameter) of the particles in a population of particles. The diameter of an essentially spherical particle may refer to the physical or hydrodynamic diameter. The diameter of a non-spherical particle usually refers to the hydrodynamic diameter. As used herein, the diameter of a non-spherical particle may refer to the largest linear distance between two points on the surface of the particle. Mean particle size can be measured using methods known in the art, such as scanning electron microscopy, dynamic light scattering, or transmission electron microscopy.

“Minimal toxicity” or “substantially no toxicity”, as used herein, refers to substantially similar inflammatory, histologic and/or visual characteristic(s) in the treated eye when compared to the same characteristic(s) in a control eye. The control eye may be an untreated healthy eye, an untreated eye of the same subject. The terms “minimal” or “substantially no toxicity” may also refer to a significantly reduced inflammatory, histologic and/or visual characteristic(s) in the treated eye when compared to the same characteristic(s) in an eye treated with the same composition by intravitreal (IVT) injection. Toxicity or inflammation can be determined by measuring the effect of the administered therapeutic agent on the intraocular pressure and/or electroretinogram of the treated eye over a period of time and comparing the measurements to those of a control eye. Differences, in these measures, between treated and control eyes of less than 30%, 25%, 20%, 15%, 10%, 9%, 8%, 7%, 6%, 5%, 4%, 3%, 2%, or 1% would be considered “minimal toxicity” or “substantially no toxicity.” In some instances, the intraocular pressure and/or electroretinogram measurements are performed one or more times (such as two or three times) per day over the over a time period stated herein. Details of measuring intraocular pressure and/or performing electroretinography measurements are described in the Examples below. Histopathology can also be used to show toxicity.

“Pharmaceutically Acceptable,” as used herein, refers to compounds, carriers, excipients, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

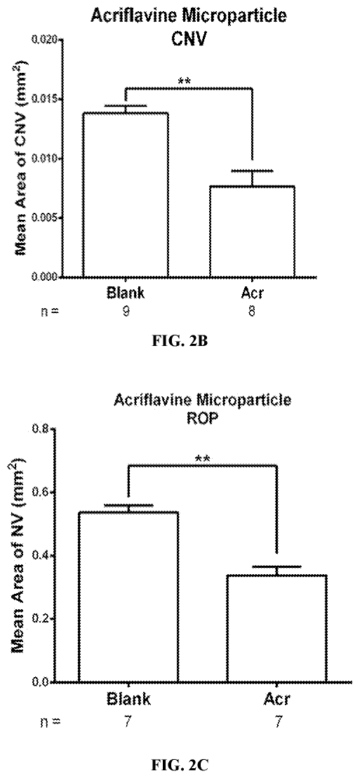

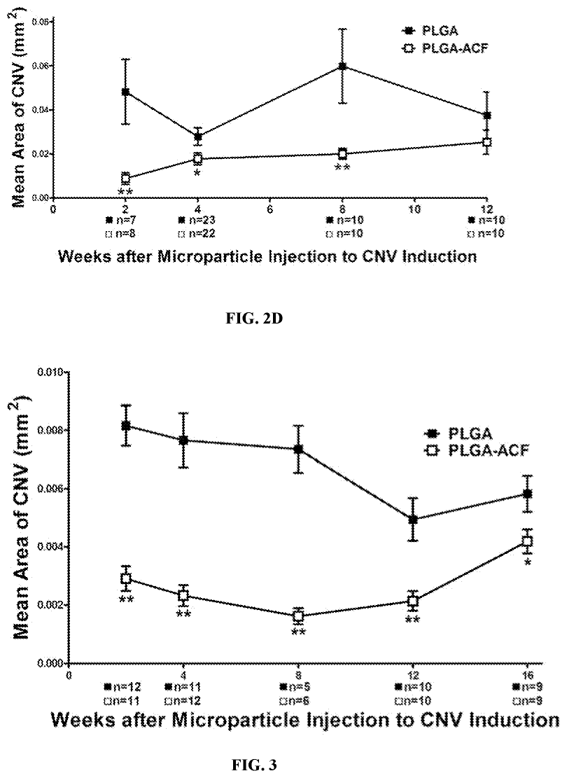

The suprachoroidal space (“SCS”) is a narrow zone between the choroid and the sclera with a potential space extending from the limbus to the optic nerve. While the innermost layer of the choroid, Bruch's membrane, is well defined, the outer border close to the sclera is a transition zone, consisting of several fibrous lamellae with variable thickness. This is shown in FIG. 2A.

Suprachoroidal injection injects drug into the suprachoroidal space (SCS) of the eye to deliver drug to posterior-segment tissues in high bioavailability and with access to structures such as the retinal vasculature and the choroidal neovascular membrane. Up to one mL of fluid is accommodated in the space, which is larger than what is required for achieving therapeutic levels that are clinically relevant for drugs. Injections of 10 to 50 μL into the SCS are well tolerated with a low risk of ocular complications. Fluid, with or without drug or particles, injected via the SCS spreads around the globe both on top of and through the choroid, distributing through the choroid and the retina. In contrast, when the same drug is injected into the vitreous, the drug spreads diffusely across all parts of the eye.

Use of the term “about” is intended to describe values either above or below the stated value in a range of approx. +/−10%; in other embodiments the values may range in value either above or below the stated value in a range of approx. +/−5%.

### II. Particles for Controlled Delivery of Therapeutic Agents

### A. Polymeric Particles

Polymeric particles for controlled release of agent typically include a biocompatible biodegradable polymer and a therapeutic, prophylactic, and/or diagnostic agent(s). The polymeric microparticles may include a coating or may be uncoated.

As used herein, “extended release” or “sustained release” refers to release of an agent at a therapeutically, prophylactically or diagnostically effective lever from particles over a period of time, which may be days, weeks, or months. Typically, release will be achieved over a period of one or more weeks following injection, preferably with no or substantially minimal, initial burst release, for example, of not more than about 5%, about 10%, about 15%, about 20%, about 25%, about 30%, about 35%, about 40%, about 45%, or about 50% of the agent loaded in the particles within one day in an isotonic solution at 37° C. Release kinetics with an initial burst of greater than about 25-50% of the agent loaded in the particles are less desirable.

### 1. Polymers

The particles are formed of biocompatible polymers. Preferably the polymers degrade by hydrolysis although enzymatically degradable polymers may be used. Preferred polymers are synthetic, but may be naturally occurring. Polymers may be homopolymers, copolymers, block copolymers, or blends thereof.

Representative synthetic polymers of the particles include hydrophobic bioerodible polymers including polyanhydrides, poly(hydroxy acids), and polyesters, as well as blends and copolymers thereof. Representative bioerodible poly(hydroxy acids) and copolymers thereof include poly(lactic acid), poly(glycolic acid), and copolymers thereof, such as poly(lactic-co-glycolic acid) also known as poly(lactic acid-glycolic acid). Other polymers include poly(hydroxy-butyric acid), poly(hydroxyvaleric acid), poly(caprolactone), poly(lactide-co-caprolactone), polyorthoesters, polyhydroxyalkanoates, polyurethanes, polyureas, poly(urea ester)s, polyamides, poly(ester amide)s, blends, and co-polymers thereof.

The polymers may be covalently bound to or mixed with other polymers such as poly(alkylene glycols), such as poly(ethylene glycol); poly(alkylene oxides) such as poly(ethylene oxide), and poly(alkylene terephthalates) such as poly(ethylene terephthalate).

Most preferred polymers are polyanhydrides and polyhydroxy acids, especially poly(lactic acid-glycolic acid) copolymers. These can be selected to provide optimal incorporation and release of drug.

The particles may include a functional group, e.g., a carboxyl or an ester group. Other functional groups include, but are not limited to, sulfhydryl, hydroxyl, and/or amino groups. The functional groups can be available, for example, for drug binding (covalent or electrostatic) or for other desired purposes.

### 2. Coatings

The microparticles may include a hydrophilic or amphiphilic coating. Amphiphilic molecules have and hydrophobic regions. The coating can be disposed on the surface of the particle, for example by bonding, adsorption or by complexation. The coating can also be intermingled or dispersed within the polymer forming the core of the particle, so that the hydrophilic ends will orient to the surface of the particles.

In a preferred embodiment, the particles have a coating formed of a polyaklyene oxide, e.g., polyoxyethylene (PEO), also referred to herein as polyethylene glycol; or polyoxypropylene (PPO), also referred to herein as polypropylene glycol (PPG), and can include co-polymers of more than one alkylene oxide. The copolymers can be, for example, random copolymers, block copolymers or graft copolymers. In some embodiments, the coating includes a polyoxyethylene-polyoxypropylene copolymer, e.g., block copolymers of ethylene oxide and propylene oxide (i.e., poloxamers). These poloxamers are available under the trade name PLURONIC® (available from BASF, Mount Olive, N.J.) and correspond to PLURONIC® F-68, F-87, F-108, and F-127, respectively.

It has been discovered that this coating is not essential to reduce toxicity when the particles are administered suprachoroidally.

### B. Therapeutic, Prophylactic, or Diagnostic Agents

The compositions typically contain a therapeutic, prophylactic, and/or a diagnostic agent. The agent may be adsorbed, encapsulated, entangled, embedded, incorporated, bound to the surface, or otherwise associated with the particle, but is preferably entrapped in the polymer matrix. The therapeutic, prophylactic, and/or a diagnostic agent may be a small molecule, a peptide, a nucleic acid, or a combination thereof.

Preferred agents are those that show toxicity or inflammation when administered intravitreally at the same dosage as in the particles. For purposes of comparing toxicity or inflammation, the agents administered intravitreally can be associated with particles (encapsulated and/or on the surface) or administered without particles. Preferably, when associated with particles, the particles are the same as those that are administered suprachoroidally. Toxicity or inflammation can be determined as described herein.

Representative therapeutic agents include immunomodulators (anti-inflammatories, immunosuppressants), antimicrobials, anti-angiogenesis agents, anti-neoplastic agents and/or combinations thereof. More than one agent can be delivered in the same particle, or a mixture of particles containing different agents may be co-formulated.

The agent may be a low molecular weight agent having a molecular weight of 1500 g/mole or 1000 g/mole or less, or a biological, such as peptides or proteins, or nucleic acid molecules. Examples include hormones, growth factors, antibody fragments, signaling molecules, and synthetic and natural nucleic acids (including RNA, anti-sense RNA, inhibitory RNA (RNAi), and oligonucleotides), and biologically active portions thereof.

Therapeutic, prophylactic or diagnostic agents to be delivered via the suprachoroidal route are those where there is greater safety and/or efficacy via this route than when administered intravitreally.

### 1. HIF-1 and HIF-2 Inhibitors

HIF-1 is a master regulator of hypoxia-induced gene expression. HIF-2 is a transcription factor that is highly homologous to HIF-1 and it also contributes to hypoxia-regulated gene expression. Typically, the HIF-1 and HIF-2 inhibitor inhibits the transcriptional activity of HIF-1, HIF-2, or both HIF-1 and HIF-2. Compositions containing one or more HIF-1 and HIF-2 inhibitors provide improved efficacy, durability, and safety for treating ocular neovascularization (“NV”). “HIF inhibitor,” “HIF-1 inhibitor,” “HIF-2 inhibitor,” as used herein, refers to, a drug that reduces the level of HIF-1 and/or its ability to stimulate the transcription of genes that contain a hypoxia response element in their promoter region.

Particles may include one or more HIF-1, HIF-2, or both HIF-1 and HIF-2 inhibitors for treatment of vascular disorders. HIF-1 and HIF-2 inhibitors are known in the art. Examples include digoxin, doxorubicin, daunorubicin, acriflavine, rapamycin, rotenone, ouabain, proscillaridin A, digitoxin, acetyldigitoxin, convallatoxin, peruvoside, strophanthin K, nerifolin, cymarin, periplocymarin, EZN-2968, irinotecan, EZN-2208, topotecan, PX-478, 2-methoxyestradio, KC7F2, glyceollins, CAY10585, 17-AAG (17-(Allylamino)-17-demethoxygeldanamycin), 17-DMAG, bisphenol A, BAY 87-2243, cryptotanshinone, vorinostat, LW6, HIF-1α inhibitor with Chemical Abstracts Service (CAS) No. 934593-90-5, PX-12, TAT-cyclo-CLLFVY (SEQ ID NO:1; CAS No. 1446322-66-2), TC-S7009, PT2385, echinomycin, indenopyrazole 21, FM19G11, YC-1, and NSC 607097, and salts, prodrugs, and derivatives thereof (Zhang et al., PNAS, 105(50): 19579-19586 (2008); Yu et al., Yonsei Med J, 58(3):489-496 (2017)).

HIF-1 inhibitors are strong suppressors of both choroidal NV at Bruch's membrane rupture sites and ischemia-induced retinal NV. The HIF-1 inhibitors digoxin, doxorubicin, daunorubicin, and acriflavine are strong inhibitors of retinal and choroidal NV, but at high doses they reduce retinal ERG function. Acriflavine, ouabain proscillaridin A, and digoxin inhibit the transcriptional activity of HIF-1 and HIF-2 at nanomolar (nM) concentrations in vitro (Zhang et al., PNAS, 105(50): 19579-19586 (2008); Yu et al., Yonsei Med J, 58(3):489-496 (2017)).

Treatments that inhibit both HIF-1 and HIF-2 may have greater benefits than those that target only HIF-1. Acriflavine (ACF) binds to HIF-1α and HIF-2α, preventing dimerization to HIF-1β, and thereby inhibits both HIF-1 and HIF-2. ACF strongly suppresses choroidal and retinal NV, but small molecules are cleared rapidly from the eye so that more frequent administration of ACF is required compared with the much larger anti-VEGF proteins that are currently used in clinical practice. Safe and sustained delivery of ACF, or other HIF-1 and HIF-2 inhibitors, is provided for treating retinal and choroidal vascular diseases.

Therapeutic, prophylactic or diagnostic agents include acriflavine (3,6-diamino-10-methylacridinium chloride mixed with 3,6-acridinediamine), a mixture of two closely related acridine molecules and derivatives of acriflavine such as proflavin, proflavine hemisulfate, and proflavine hydrochloride, and other prodrugs or salts thereof.

Acriflavine (ACF) is a mixture of trypaflavine (TRF) and proflavine (PRF). ACF is known for its trypanocidal, antibacterial, and antiseptic activity and is mostly used topically for wound healing as well as systemically for gonorrhea treatment via both intravenous and oral administration. Systemic use, however, is extremely limited due to toxicity.

The effects of ACF on cancer cells were first reported 50 years ago, but interest again peaked in 2009, when ACF, out of 336 FDA-approved drugs, was shown to be the most potent hypoxia-inducible factor 1a (HIF-1α) inhibitor. ACF was shown to inhibit HIF-1α transcriptional activity by preventing the dimerization of HIF-1α and HIF-1β, which reduced angiogenic cells mobilization and tumor vascularization and successfully suppressed tumor growth in prostate cancer xenografts (Lee et al, Proc Natl Acad Sci USA, 106 (42):17910-7915(2009)). Studies have shown that HIF-1α inhibition is not its only mechanism of action but that ACF can also induce apoptotic and autophagic effects in cancer cells (Fan et al., Tumour Biol; 35(10):9571-9576 (2014)). ACF has demonstrated highly effective antitumor activity against a wide spectrum of cancers (Hassan et al., Cancer Science, 102(12): 2206-213 (2011); Lee et al., Anticancer Res. 34(7):3549-3556 (2014)).

Acriflavine inhibits Vascular Endothelial Growth Factor (VEGF), which is downstream from HIF-1. HIF-1 also helps sustain a glycolytic phenotype, so inhibiting it may also facilitate the switch from glycolysis back to mitochondrial respiration, thwarting the Warburg Effect—the production of energy by high rate of glycolysis followed by lactic acid fermentation in the cytosol (Alfarouk et al, Oncoscience, 1(14):777-802 (2014)). Acriflavine interferes with the binding domain of HIF-2 thereby preventing its downstream angiogenic and glycolytic factors from functioning. Additionally, the inhibition of HIF-2 has been shown to promote the p53 axis, which reduces the glycolytic phenotype (Bertout et al., Proc Natl Acad Sci USA, 106:14391-14396 (2009)).

Acriflavine also inhibits topoisomerases I and II (Hassan et al., Cancer Science, 102(12): 2206-213 (2011)). These enzymes are involved in DNA coiling during replication. Cancer drugs that target topoisomerase II are known to create DNA damage in tumor cells during replication (Nitiss, Nature Reviews Cancer, 9(5); 338-350 (2009)). Acriflavine also inhibits protein kinase c (PKC), which plays a key role in cell proliferation pathways (Kim et al., Drug Metabolism and Disposition, 26(1):66-72 (1998)).

The particles may include other therapeutic agents, for example, anti-inflammatory agents, antimicrobial agents, anti-angiogenic agents, chemotherapeutic agents, and combinations thereof.

### 2. Imaging Agents

Imaging agents may also be incorporated in the particles. The imaging agents include one or more radionuclides, optical tracers such as bioluminescent, chemiluminescent, fluorescent or other high extinction coefficient or high quantum yield optical tracers, T1 magnetic resonance imaging (MRI) agents in the class of heavy metals (gadolinium, or dysprosium), T2 contrast agents (iron oxide, or manganese oxide), or iodinated agents.

### C. Size and Loading

The particles preferably have an average population diameter of, for example, between about 100 nm and about 100 μm, between about 1 μm and about 80 μm, between about 1 μm and about 70 μm, between about 1 μm and about 60 μm, between about 1 μm and about 50 μm, between about 1 μm and about 40 μm, between about 1 μm and about 30 μm, between about 1 μm and about 20 μm, preferably between about 2 μm and about 80 μm, more preferably between about 1 μm and about 40 μm, most preferably between about 1 μm and about 20 μm. The average diameter can be measured by using scanning electron microscopy, dynamic light scattering, or transmission electron microscopy. A preferred method to determine the average diameteriseasured using scanning electron microscopy.

The particles may be loaded with the therapeutic, prophylactic, or diagnostic agent at a loading between about 1% and about 30% weight of the agent to the weight of the particle (w/w), such as between about 1% and about 18% (w/w), about 1% and about 16% (w/w), about 1% and about 14% (w/w), about 1% and about 12% (w/w), or about 1% and about 10% (w/w). Exemplary loadings include about 1% (w/w), about 2% (w/w), about 3% (w/w), about 4% (w/w), about 5% (w/w), about 6% (w/w), about 7% (w/w), about 8% (w/w), about 9% (w/w), about 10% (w/w), about 15% (w/w), about 20% (w/w), about 25%, or about 30% (w/w).

Typically, the therapeutic, prophylactic, or diagnostic agent in the particles is at a concentration between about 10 μg/mg and about 200 μg/mg particles, such as between about 10 μg/mg and about 180 μg/mg, about 10 μg/mg and about 160 μg/mg, about 10 μg/mg and about 140 μg/mg, about 10 μg/mg and about 120 μg/mg, or about 10 μg/mg and about 100 μg/mg of particles. Exemplary concentrations include about 10 μg/mg, about 20 μg/mg, about 30 μg/mg, about 40 μg/mg, about 5μg/mg, about 60 μg/mg, about 70 μg/mg, about 80 μg/mg, about 90 μg/mg, about 100 μg/mg, about 120 μg/mg, about 140 μg/mg, about 160 μg/mg, about 18 μg/mg, or about 20 μg/mg.

### D. Pharmaceutically Acceptable Excipients

The particles may be included in compositions containing one or more pharmaceutically acceptable excipients. Pharmaceutically acceptable excipients for injection may include, but are not limited to, diluents, preservatives, surfactants, emulsifiers, emulsion stabilizers, anti-oxidants, preservatives, and pH modifying agents.

Injectable formulations can be prepared as suspensions or in solid forms suitable for preparing solutions or suspensions upon the addition of a reconstitution medium prior to injection. Acceptable solvents include, for example, water, Ringer's solution, phosphate buffered saline (PBS), and isotonic sodium chloride solution. Generally, dispersions are prepared by incorporating the sterilized particles into a sterile vehicle which contains the basic dispersion medium and the required other ingredients from those listed above. In the case of sterile powders for the preparation of sterile injectable solutions, the preferred methods of preparation are vacuum-drying and freeze-drying techniques which yield a powder plus any additional desired ingredient from a previously sterile-filtered solution thereof.

In some instances, the formulation is distributed or packaged in a liquid form. Alternatively, formulations can be packed as a solid, obtained, for example by lyophilization of a suitable liquid formulation. The solid can be reconstituted with an appropriate carrier or diluent prior to administration.

### III. Methods of Making

The particles can be fabricated using methods known in the art including emulsion, nanoprecipitation, microfluidics, solvent evaporation, solvent extraction, phase inversion or spray drying.

In some embodiments, a particle is prepared using an emulsion solvent evaporation method. A polymeric material is dissolved in a water immiscible organic solvent and mixed with an agent solution or a combination of agent solutions. In some embodiments a solution of a therapeutic, prophylactic, or diagnostic agent to be encapsulated is mixed in solid form with the polymer solution. The polymer is dissolved in a volatile organic solvent, such as methylene chloride. A substance to be incorporated is added to the solution, and the mixture is suspended in an aqueous solution that contains a surface active agent such as poly(vinyl alcohol). The resulting emulsion is stirred until most of the organic solvent evaporated, leaving solid microspheres. Microspheres with different sizes (1-1000 microns) and morphologies can be obtained by this method. Methods for forming microspheres using solvent evaporation techniques are well known.

In another embodiment, particles are prepared using nanoprecipitation methods or microfluidic devices. A polymeric material is mixed with a drug or drug combinations in a water miscible organic solvent.

Methods of making nanoparticles using microfluidics are known in the art. Suitable methods include those described in U.S. Patent Application Publication No. 2010/0022680 A1 by Karnik et al.

Solvent extraction to make particles is also well known. The substance to be incorporated is dispersed or dissolved in a solution of the selected polymer in a volatile organic solvent like methylene chloride. This mixture is suspended by stirring in an organic oil, such as silicon oil, to form an emulsion. Microspheres that range between 1-300 microns can be obtained by this procedure.

Methods for forming microspheres using spray drying techniques are well known. The polymer is dissolved in an organic solvent such as methylene chloride. A known amount of a substance to be incorporated is suspended (insoluble agent) or co-dissolved (soluble agent) in the polymer solution. The solution or the dispersion then is spray-dried. Microspheres typically ranging between 1-10 microns are obtained.

Microspheres can be formed from polymers using a phase inversion method wherein a polymer is dissolved in a good solvent, fine particles of a substance to be incorporated, such as a drug, are mixed or dissolved in the polymer solution, and the mixture is poured into a strong non-solvent for the polymer, to spontaneously produce, under favorable conditions, polymeric microspheres, wherein the polymer is either coated on the particles or the particles are dispersed in the polymer. The method can be used to produce microparticles in a wide range of sizes, including, for example, about 100 nanometers to about 10 microns.

### IV. Methods of Using

### A. Disorders and Diseases to be Treated

The particles and/or compositions are suitable for treating eye diseases, particularly ocular neovascularization. Ocular neovascularization includes retinal and choroidal vascular diseases (Campochiaro, J Mol Med (Berl), 91(3):311-321 (2013)).

Retinal and choroidal vascular diseases constitute the most common causes of moderate and severe vision loss in developed countries. They can be divided into retinal vascular diseases, in which there is leakage and/or neovascularization (NV) from retinal vessels, and subretinal NV, in which new vessels grow into the normally avascular outer retina and subretinal space. The first category of diseases includes diabetic retinopathy, retinal vein occlusions, and retinopathy of prematurity and the second category includes neovascular age-related macular degeneration (AMD), ocular histoplasmosis, pathologic myopia, and other related diseases.

Retinal hypoxia is a key feature of the first category of diseases resulting in elevated levels of hypoxia-inducible factor-1 (HIF-1) which stimulates expression of vascular endothelial growth factor (VEGF), platelet-derived growth factor-B (PDGF-B), placental growth factor, stromal-derived growth factor-1 and their receptors as well as other hypoxia-regulated gene products such as angiopoietin-2.

Although hypoxia has not been demonstrated as part of the second category of diseases, HIF-1 is elevated and thus the same group of hypoxia-regulated gene products plays a role. Clinical trials have shown that VEGF antagonists provide major benefits for patients with subretinal NV due to AMD and even greater benefits are seen by combining antagonists of VEGF and PDGF-B.

Therapeutic strategies include directly targeting HIF-1, or HIF-1 and HIF-2, as anti-angiogenic treatments. HIF-1 is a master regulator of hypoxia-induced gene expression that is increased in ischemic retina and upregulates multiple angiogenic proteins and their receptors, including vascular endothelial growth factor-A (VEGF) and its receptors. It also appears that retinal hypoxia contributes to progression of background diabetic retinopathy, because wide angle fluorescein angiography shows correlation between progression of nonperfusion and worsening of diabetic retinopathy.

Macular degeneration (MD) is a chronic eye disease that occurs when tissue in the macula, the part of the retina that is responsible for central vision, deteriorates. Degeneration of the macula causes blurred central vision or a blind spot in the center of your visual field. Macular degeneration occurs most often in people over 60 years old, in which case it is called Age-Related Macular Degeneration (ARMD) or (AMD). AMD is the leading cause of blindness in the United States and many European countries. About 85-90% of AMD cases are the dry, atrophic, or nonexudative form, in which yellowish spots of fatty deposits called drusen appear on the macula. The remaining AMD cases are the wet form, so called because of leakage into the retina from newly forming blood vessels in the choroid, a part of the eye behind the retina. Normally, blood vessels in the choroid bring nutrients to and carry waste products away from the retina. Sometimes the fine blood vessels in the choroid underlying the macula begin to proliferate, a process called choroidal neovascularization (CNV). When those blood vessels proliferate, they leak, causing damage to cells in the macula often leading to the death of such cells. The neovascular “wet” form of AMD is responsible for most (90%) of the severe loss of vision. There is no cure available for “wet” or “dry” AMD.

The exact causes of AMD are not known, however, contributing factors have been identified. Factors that contribute to AMD include reactive oxidants which cause oxidative damage to the cells of the retina and the macula, high serum low density cholesterol lipoprotein (LDL) concentration, and neovascularization of the choroid tissue underlying the photoreceptor cells in the macula.

Treatments for wet AMD include photocoagulation therapy, photodynamic therapy, and transpupillary thermotherapy. AMD treatment with transpupillary thermotherapy (TTT) photocoagulation is a method of delivering heat to the back of the patient's eye using an 810 nm infrared laser, which results in closure of choroidal vessels. AMD treatment with photocoagulation therapy involves a laser aimed at leakage points of neovascularizations behind the retina to prevent leakage of the blood vessel. Photodynamic therapy (PDT) employs the photoreactivity of a molecule of the porphyrin type, called verteporphin or Visudyne, which can be performed on leaky subfoveal or juxtafoveal neovascularizations. Pegaptanib sodium injection (MACUGEN®) is an FDA approved drug that inhibits abnormal blood vessel growth by attacking a protein that causes abnormal blood vessel growth.

Other potential treatments for “wet” AMD that are under investigation include angiogenesis inhibitors, such as anti-VEGF antibody, and anti-VEGF aptamer (NX-1838). Integrin antagonists to inhibit angiogenesis has also been proposed, and PKC412, an inhibitor of protein kinase C. Cytochalasin E (Cyto E), a natural product of a fungal species that inhibits the growth of new blood vessels is also being investigated to determine if it will block growth of abnormal blood vessels in humans. The role of hormone replacement therapy is being investigated for treatment of AMD in women.

There are no treatments available to reverse “dry” AMD. Treatments shown to inhibit progression of AMD include supplements containing antioxidants. The use of a gentle “sub-threshold” diode laser treatment that minimizes damage to the retina is being investigated for treatment of “dry” AMD. Another potential treatment for AMD includes rheopheresis, which is a form of therapeutic blood filtration that removes “vascular risk factor” including LDL cholesterol, fibrinogen, and lipoprotein A.

### B. Evaluation of Safety and Therapeutic Efficacy

The particulate compositions described herein typically provide therapeutic or prophylactic benefit without causing substantial toxicity when administered subconjunctivally. Toxicity of the particle compositions can be determined as described above. The dose ratio between toxic and therapeutic (or prophylactic) effect is the therapeutic index. Compositions that exhibit high therapeutic indices are preferred.

The therapeutic effect and safety may be shown by examining intraocular pressure (IOP) of the treated eye, the presence, reduction, or absence of inflammation in the treated eye, or by imaging the fundus of the treated eye. Inflammation and IOP can be measured after administration of the compositions either intravitreally or subconjunctivally, and then compared. For example, a Reichert Tono-Pen contact tonometer can be used to assess the IOP, and changes in IOP, in an eye, for example the anterior chamber (AC). The subjects are evaluated on several post-injection days, for example at 1, 7, 14 and 30 days post-injection using the same evaluation procedure on each day. One or more measurements, for example about five, can be obtained for each subject at each time point. When measuring IOP according to methods known in the art, for example the method described above, IOP is shown to be reduced by, for example, at least about 10%, or at least about 30%, or at least about 50% over what is observed in an untreated eye, in the absence of a composition.

To measure inflammation, Slit-lamp biomicroscopy can be performed to examine an eye compartment such as the AC, for signs of inflammation. The examination can involve observation of criteria such as the presence of cells, flare and fibrin. The subjects can be evaluated on several post-injection days, for example at 1, 7, 14 and 30 days post-injection using the same evaluation procedure on each day. After general and/or local anesthesia is achieved, each subject can be examined for gross abnormalities. The exams can be performed by the same trained ophthalmologist, and the ophthalmologist can be blinded to the assignment of the treatment and control subjects. Quantification of inflammation of an eye compartment such as the AC can be performed using a modified version of the Standard Uveitis Nomenclature clinical grading scheme, as detailed more fully in the examples.

Fundus imaging typically uses a fundus camera to record color images of the condition of the interior surface of the eye, in order to document the presence of disorders and monitor their change over time. A fundus camera or retinal camera is a specialized low power microscope with an attached camera designed to photograph the interior surface of the eye, including the retina, retinal vasculature, optic disc, macula, and posterior pole (i.e. the fundus). The retina is imaged to document conditions such as diabetic retinopathy, age related macular degeneration, macular edema and retinal detachment. Fundus photography is also used to help interpret fluorescein angiography as certain retinal landmarks visible in fundus photography are not visible on a fluorescein angiogram. The eyes will be dilated before the procedure. Widening (dilating) a patients pupil increases the angle of observation. This allows imaging a much greater area and have a clearer view of the back of the eye.

When comparing an IOP or inflammation, the measurements compared can, and frequently should, be taken at similar time points post injection. For example, an IOP measurement for a coated particle taken 7 days post-injection is generally compared with an IOP measurement for an uncoated particle taken 7 days post-injection

The compositions typically have minimal or no negative effect on vision. Typically, minimal or no negative effect on vision may be illustrated by obtaining substantially similar visual characteristics from the treated eye as from a control eye.

Vision and visual characteristics may be assessed by routine examination by the ophthalmologist, optician, or a veterinary care staff. In other instances, the vision may be assessed by minimally invasive methodologies, including electroretinogram recordings.

The vision and/or visual characteristics of the treated eye may be compared to those of a control eye. The control eye may be an untreated eye, the untreated eye of the same subject, or the untreated eye of the same subject prior to treatment.

### C. Methods and Devices for Suprachoroidal Administration

The particles and compositions are administered by injection of an effective amount of the therapeutic agent to the suprachoroidal space (SCS) of the eye.

Methods for SCS injections with microneedles are known and have been used to deliver nanoparticles and microparticles to the eye (Patel et al., IOVS, 53(8):4433-4441 (2012); Patel et al., Pharm Res 28:166-176 (2011)). Typically, these microneedles have an internal diameter of the needle of about 110 μm or less. Injection can be achieved using a syringe or cannula and needle such as a 30-34 G needle, but specialized devices for suprachoroidal administration are now available. These include the CLEARSIDE BIOMEDICAL Microinjector and transdermal microneedle patches that are in development.

In the preferred embodiment, compositions are injected with a single needle. The needle is typically at least 34 gauge, but may be of lower gauge, such as 33 gauge, 32 gauge, 31 gauge, 30 gauge, 29 gauge, 28 gauge, 27 gauge, 26 gauge, or 25 gauge. Typically, the needle has an internal lumen diameter of at least about 80 μm, or between 82 μm and 260 μm.

In one embodiment, the eye is anesthetized using drops and a subconjunctival injection, and sterilized with aseptic compound drops. Four mm posterior to the limbus in any quadrant of the eye, the flat portion of a trocar blade is touched to the surface of the conjunctiva with the tip of the blade directed parallel to the limbus. With the blade lying flat on the conjunctiva, it is gradually advanced with the tip of the blade oriented just slightly downward so that the blade penetrates the conjunctiva and then the sclera at a very oblique angle. The trocar/cannula is advanced slowly until a portion of the cannula is within the eye and the remainder with the hub is lying on its side on the surface of the eye. The trocar is slowly removed by holding the cannula adjacent to the hub with a forceps while slowly removing the trocar. The tip of the cannula is then sitting in the suprachoroidal space. A primed 30-gauge needle attached to a syringe containing 50 μl of fluid is inserted through the valves into the cannula and the contents are slowly injected into the suprachoroidal space. As the contents are injected, the intraocular pressure increases limiting the volume that could be injected. Typically, 50 μl is a safe limit for injection into a human eye, because with that volume the increase in intraocular pressure is not sufficient to close the retinal circulation, however it would be prudent to examine the retinal circulation by indirect ophthalmoscopy after injection.

A major advantage of the cannula system is that it can remain in the suprachoroidal space for a prolonged period of time, allowing the intraocular pressure to return to normal, making a second injection of 50 μl possible. Alternatively, after the first injection, a 30 gauge needle can be inserted into the anterior chamber and 100-200 μl of aqueous humor can be withdrawn. A second suprachoroidal injection can then be given with volume even larger than the first (up to 150 μl if 200 μl of aqueous was removed). Another alternative is to remove 100-200 μl of aqueous immediately after the cannula is inserted but before suprachoroidal injection. This allows a single suprachoroidal injection of up to 200 μl.

Thirty seconds after the final suprachoroidal injection, a cotton tip is held over the cannula entry site, the cannula is slowly withdrawn, and the cotton tip held in place for 30 seconds.

### D. Cumulative Therapeutic Dose

The compositions are typically administered at a cumulative therapeutic dose of the agent of at least about 10 μg/mg of the particles. Cumulative therapeutic dose refers to the total amount of the agent in the composition at the time of administration. Typically, the cumulative therapeutic dose is sufficient to be therapeutic throughout the period of controlled release of the agent following single administration.

The therapeutic, prophylactic, or diagnostic agent may be present in the particles is a cumulative therapeutic dose between about 10 μg/mg and about 200 μg/mg microparticles, such as between about 10 μg/mg and about 180 μg/mg, about 10 μg/mg and about 160 μg/mg, about 10 μg/mg and about 140 μg/mg, about 10 μg/mg and about 120 μg/mg, or about 10 μg/mg and about 100 μg/mg microparticles.

Exemplary cumulative therapeutic doses include about 10 μg/mg, about 20 μg/mg, about 30 μg/mg, about 40 μg/mg, about 50 μg/mg, about 60 μg/mg, about 70 μg/mg, about 80 μg/mg, about 90 μg/mg, about 100 μg/mg, about 120 μg/mg, about 140 μg/mg, about 160 μg/mg, about 18 μg/mg, or about 20 μg/mg microparticles.

Compositions containing a cumulative therapeutic dose of an agent may be administered at a frequency of less than once about every 8 weeks, once about every 9 weeks, once about every 10 weeks, once about every 11 weeks, once about every 12 weeks, once about every 13 weeks, once about every 14 weeks, once about every 6 months, once about every 9 months, or once about every one year.

The particles and compositions provide sustained release of a therapeutic agent over a period of time between about two weeks and one year. The release kinetics from the compositions typically do not, but may include, a small initial burst release. The compositions typically reach a steady rate of agent release within about two days following administration.

Steady rate of release refers to an amount of an agent released within a specified time period and repeated over a longer time period. For example, a steady rate of release may be release of about 10% of the loaded weight of the agent over a period of one week, two weeks, three weeks, four weeks, etc. The duration of release at a steady rate may be, for example for at least about 2 weeks, for at least about 4 weeks, for at least about 6 weeks, for at least about 7 weeks, for at least about 8 weeks, for at least about 9 weeks, for at least about 10 weeks, for at least about 11 weeks, or for at least about 12 weeks, or for longer periods of time, in an isotonic solution at 37 ° C. or in vivo

Preferably, the particles and/or compositions are administered not more frequently than once about every 6 weeks, once about every 8 weeks, once about every 16 weeks, once about every 20 weeks, or once about every 24 weeks.

Typically, the compositions provide a therapeutic dose between about 0.1 nM and about 200 nM within the eye or eye compartment, per week, preferably between about 0.1 nM and about 150 nM per week, more preferably between about 0.1 nM and about 100 nM per week, most preferably between about 0.1 nM and about 10 nM per week.

The agent may be released at a steady rate at a therapeutic dose between about 0.1 nM and about 10 nM per week, such as between about 0.1 nM and about 2 nM per week and/or between about 1 nM and about 10 nM per week.

The present invention will be further understood by reference to the following non-limiting examples.

### EXAMPLES

### Example 1. Preparation and Characterization of PLGA-ACF Microparticles (MPs)

Acriflavine (ACF) is highly water soluble, which makes achieving high drug loading and sustained release from hydrophobic, biodegradable polymeric particles challenging. Past reports of loading ACF into MPs, lipid vesicle creams, and wafers described rapid burst release in just minutes to hours in vitro.

### Materials and Methods

### Preparation of PLGA-ACF MPs

PLGA-ACF MPs were prepared using a single emulsion solvent evaporation method. Parameters that were varied include (i) the PLGA polymer end group (carboxylic vs. ester), (ii) polymer molecular weight (PLGA1A, 2A, 7A), (iii) polymer concentration (50-200 mg/ml), and (iv) the pH of the water phase (5.0, 6.8, 7.4, 9.0).

Different methods for preparing the PLGA-ACF MPs were tested.

### Method 1

200 mg PLGA 1A was dissolved in 4 mL methylene chloride, 40 mg acriflavine was dissolved in 0.5 mL DMSO, then mixed together, homogenized at 5000 rpm, 1 min. The mix was poured into an aqueous solution containing 1% polyvinyl alcohol (PVA) and stirred for 2 hours. The particles were collected, washed with double distilled water, and freeze dried.

### Method 2

200 mg PLGA was dissolved in 4 mL, 2 mL, or 1 mL methylene chloride, 40 mg acriflavine was dissolved in 0.5 mL DMSO, then mixed together, and homogenized at 5000 rpm, 1 min. The mix was poured into an aqueous solution containing 1% polyvinyl alcohol (PVA) and stirred for 2 hours. The particles were collected, washed with double distilled water, and freeze dried.

### Method 3

200 mg PLGA was dissolved in 2 mL methylene chloride, 40 mg acriflavine was dissolved in 0.5 mL DMSO, these were mixed together, then triethylamine (TEA, 50, 100, 200, 400 μL) was added, and the mixture homogenized at 5000 rpm, 1 min. The mixture was poured into an aqueous solution containing 1% polyvinyl alcohol (PVA) and stirred for 2 hours. The particles were collected, washed with double distilled water, and freeze dried.

### Method 4

200 mg PLGA was dissolved in 2 mL methylene chloride, 40 mg acriflavine was dissolved in 0.5 mL DMSO, then mixed together, and homogenized at 5000 rpm for 1 min. The mix was poured into an aqueous solution with pH 5.0, 6.8, 7.4, or 9.0 containing 1% polyvinyl alcohol (PVA) and stirred for 2 hours. The particles were collected, washed with double distilled water, and freeze dried.

### Method 5

180 mg PLGA 7E or 7A and 20 mg PEG-PLGA were dissolved in 4 mL methylene chloride, 40 mg acriflavine was dissolved in 0.5 mL DMSO, then mixed together, homogenized at 5000 rpm, 1 min. The mix was poured into an aqueous solution containing 1% polyvinyl alcohol (PVA) and stirred for 2 hours. The particles were collected, washed with double distilled water, and freeze dried.

### Method 6

180 mg PLGA 4A and 20 mg PEG-PLGA were dissolved in 1 mL methylene chloride, 40 mg acriflavine was dissolved in 0.5 mL DMSO and TEA, then mixed together, homogenized at 6000 rpm, 1 min. The mix was poured into an aqueous solution containing 1% polyvinyl alcohol (PVA) and stirred for 2 hours. The particles were collected, washed with double distilled water, and freeze dried.

### Method 7

180 mg PLA 4.5E and 20 mg PEG-PLGA were dissolved in 2 mL methylene chloride, 40 mg acriflavine was dissolved in 0.5 mL DMSO and TEA, then mixed together, and homogenized at 6000 rpm, 1 min. The mixture was poured into an aqueous solution containing 1% polyvinyl alcohol (PVA) and stirred for 2 hours. The particles were collected, washed with double distilled water, and freeze dried.

### Method 8

PLGA 7A or 7E, PLGA 2A, and PEG-PLGA, at different ratios, were dissolved in 2 mL methylene chloride, 40 mg acriflavine was dissolved in 1 mL DMSO, then mixed together, and homogenized at 6000 rpm for 1 min. The mixture was poured into an aqueous solution containing 1% polyvinyl alcohol (PVA) and stirred for 2 hours. The particles were collected, washed with double distilled water, and freeze dried.

The final microparticle formulation used in animal studies was formulated by dissolving 200 mg PLGA (2A, 50:50 LA:GA) (Evonik Corporation, Piscataway, N.J.) in 2 mL of dichloromethane (DCM, Sigma-Aldrich), and mixing with 40 mg ACF (Sigma-Aldrich) dissolved in 0.5 ml dimethyl sulfoxide (DMSO, Sigma-Aldrich). The mixture was homogenized (L4RT, Silverson Machines) at 5000 RPM for 1 minute. The homogenized mixture was then poured into a solution containing 1% polyvinyl alcohol (25 kDa, Polysciences, Warrington, Pa.) in phosphate buffered saline (PBS, pH 7.4) under continuous stirring. Particles were hardened by allowing solvent to evaporate while stirring at room temperature for 2 h. Particles were collected via centrifugation (International Equipment Co) at 2,000×g for 15 min, and washed with HyPure cell culture grade water (endotoxin-free, HYCLONE™, Logan, Utah) and re-collected by centrifugation three times. The washed particles were then lyophilized and stored frozen until used. Microparticles were resuspended in a sodium hyaluronate solution (HEALON®) diluted 5-fold with endotoxin-free water at the desired concentration prior to injection.

In a specific example, acriflavine microparticles were prepared as follows. 200 mg PLGA (2A) was dissolved in 4 mL methylene chloride, 40 mg acriflavine was dissolved in 0.5 mL DMSO, and the solutions were mixed together and homogenized at 5000 rpm, for 1 min. The mix was poured into an aqueous solution containing 1% polyvinyl alcohol (PVA) in pH 7.4 PBS and stirred for 2 hours. The particles were collected, washed with double distilled water, freeze dried, and the size estimated to be about 7.3 μm with loading of 6.8% w/w (2 microgram ACF per 29.4 microgram microparticle).

### Characterization of PLGA-ACF MPs

Particle size distribution was determined using a Coulter Multisizer 4 (Beckman Coulter, Inc., Miami, Fla.). Particles were resuspended in double distilled water and added dropwise to 100 ml of ISOTON II solution until the coincidence of the particles was between 8% and 10%. At least 100,000 particles were sized for each batch of particles to determine the mean particle size and size distribution. Particle morphology was evaluated by LEO1530/Zeiss Field-emission scanning electron microscopy (SEM). Particles were lyophilized and mounted onto SEM stubs at room temperature before sputter coating with a thin layer of platinum (Denton Vacuum, LLC Technologies). To determine the drug loading, microparticles were dissolved in DMSO and the total drug content was calculated by measuring the UV absorbance at 420 nm in triplicate. Absorbance of blank particles dissolved in DMSO at the same polymer concentration was subtracted to account for polymer interference. The microparticle size was 7.3±1.8 μm and the ACF loading was 6.8% (w/w) (2 microgram ACF per 29.4 microgram microparticle).

### Preparation of ACF-PSA-PEG₃ MPs

### Conjugation of acriflavine to poly(ether-anhydrides)

Poly(ethylene glycol)3-co-poly(sebacic acid) (PEG₃SA) was synthesized by melt polycondensation. Briefly, sebacic acid was refluxed in acetic anhydride to form sebacic acid prepolymer (Acyl-SA). Citric-Polyethylene glycol (PEG₃) was prepared. Briefly, CH₃O-PEG-NH₂ 2.0 g, citric acid 25.87 mg, DCC 82.53 mg and DMAP 4.0 mg were added to 10 mL methylene chloride, stirred overnight at room temperature, then precipitated and washed with ether, and dried under vacuum. Acyl-SA and mPEG (10% w/w) were placed into a flask under a nitrogen gas blanket and melted (180° C.) and high vacuum was applied. Nitrogen gas was swept into the flask after 15 minutes. The reaction was allowed to proceed for 30 min. Polymers were cooled to ambient temperature, dissolved in chloroform, and precipitated into excess petroleum ether.

### Synthesis of poly(ether anhydride)-acriflavine Particles

PEG₃SA acriflavine microparticles were prepared by dissolving PEG₃SA with acriflavine at defined ratios in 3 mL dichloromethane and 1 mL DMSO and reacting for 2 hrs at 50° C. before homogenizing (L4RT, Silverson Machines, East Longmeadow, Mass.) into 100 mL of an aqueous solution containing 0.1% polyvinyl alcohol (25 kDa, Sigma). Particles were hardened by allowing chloroform to evaporate at room temperature while stirring for 2 hrs. Particles were collected and washed three times with double distilled water via centrifugation at 8,000×g for 15 min (International Equipment Co., Needham Heights, Mass.). Particle size distribution was determined using a Coulter Multisizer IIe (Beckman). Particles were resuspended in double distilled water and added dropwise to 100 ml of ISOTON II solution until the coincidence of the particles was between 8% and 10%. At least 100,000 particles were sized for each batch of particles to determine the mean particle size and size distribution.

### ACF Release from PLGA-ACF MPs

Release kinetics were obtained by resuspending microparticles at 5 mg/mL in 1 ml phosphate buffered saline (PBS, pH 7.4) and incubating at 37° C. on a platform shaker (140 RPM). Supernatant was collected at predetermined intervals by centrifugation at 2,000×g, or 13,500×g, for 5 min. Drug-containing supernatant was collected and particles were resuspended in 1 ml of fresh PBS. ACF concentration in the collected supernatant was assayed via absorbance at 420 nm, or 460 nm, in triplicate for each sample (n=3).

### Results

### Development of Microparticles that Provide Sustained Release of acriflavine

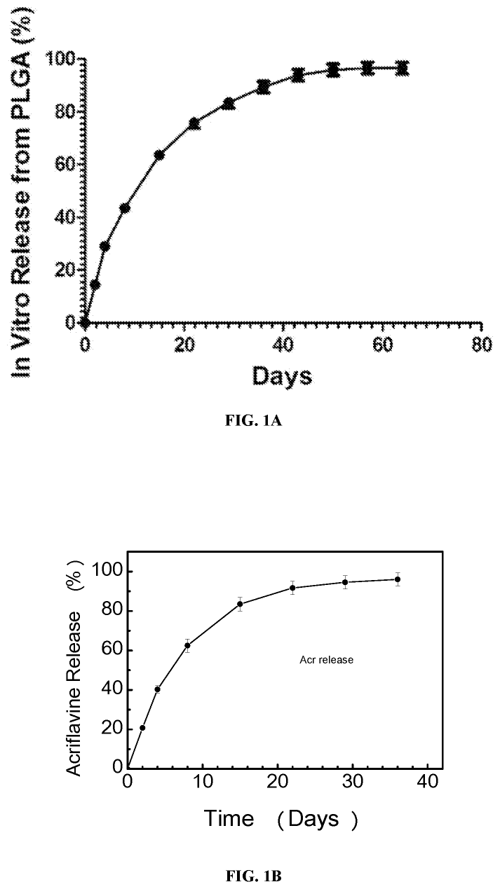

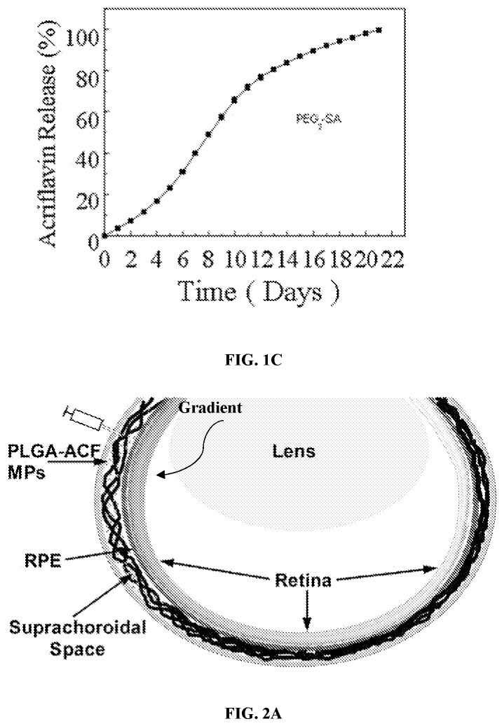

Formulation variables were modified to achieve the highest possible drug loading into MPs in the appropriate size range that can be easily administered through needle gauges that are standard for use in ophthalmic injections. Specifically, PLGA polymer end group, polymer molecular weight, polymer concentration, and the pH of the water phase were varied. Spherical PLGA-ACF MPs were obtained with an average size of 7.3±1.8 μm, which could easily be injected through a needle as small as 30G. Contrary to prior reports, there was minimal to no ACF burst release from the PLGA-ACF MPs in vitro, and the release was sustained for at least 40 days (FIG. 1A). Less than about 20% of ACF was released in the first two days.

Tables 1-14 summarize testing of the different parameters: (i) the PLGA polymer end group (carboxylic vs. ester), (ii) polymer molecular weight (PLGA1A, 2A, 7A), (iii) polymer concentration (50-200 mg/ml), and (iv) the pH of the water phase (5.0, 6.8, 7.4, 9.0). Tables 1-14 also present the results achieved with varying these different parameters. Representative kinetics of acriflavine release from the PLGA-ACF MPs prepared according to these tests are shown in FIG. 1B. As in FIG. 1A, less than about 20% of ACF was released in the first two days, showing no burst release.

FIG. 1C is a line graph showing the kinetics of acriflavine release (%) over time (days) from ACF-PSA-PEG₃ MPs. The ACF-PSA-PEG₃ MPs microparticles had an average size of 13.5 μm, loading 3.5% w/w, and in vitro release profile at pH 7.4, 37° C., showing absence of initial burst release.

TABLE 1
PLGA particles - effect of PLGA end group

| polymer | End group | Loading (%)^{a} | Size (μm) |
|---|---|---|---|
| PLGA2E | ester | 1.9 | 7.9 |
| PLGA2A | Acid | 2.4 | 7.5 |

200 mg polymer + 4 mL CH₂Cl₂ + 0.5 mL DMSO, water phase pH = 6.8
^{a}w/w

TABLE 2
PLGA particles - effect of PLGA molecular weight

| polymer | Mw (KDa) | Loading (%)^{a} | Size (μm) | Release |
|---|---|---|---|---|
| PLGA1A | 12 | 3.2 | 5.8 | FIG. 1B |
| PLGA2A | 17 | 2.4 | 7.5 | |
| PLGA7A | 113 | 1.1 | 11.4 | |

200 mg polymer + 4 mL CH₂Cl₂ + 0.5 mL DMSO, water phase pH = 6.8
^{a}w/w

TABLE 3
PLGA particles - effect of PLGA2A concentration

| Polymer (mg/mL) | Loading (%)^{a} | Size (μm) |
|---|---|---|
| 50 | 2.4 | 7.5 |
| 100 | 4.2 | 10.1 |
| 200 | 5.1 | 13.5 |

200 mg PLGA 2A, CH₂Cl₂ (4, 2, 1 mL) + 0.5 mL DMSO, water phase pH = 6.8,
^{a}w/w

TABLE 4
PLGA particles - effect of TEA

| TEA (μL) | Loading (%)^{a} | Size (μm) |
|---|---|---|
| 50 | 4.8 | 12.9 |
| 100 | 6.8 | 16.1 |
| 200 | 6.1 | 17.8 |
| 400* | Particles broken | |

200 mg PLGA 2A, +2 mL CH₂Cl₂ + DMSO 0.5 mL water phase pH = 6.8,
^{a}w/w

TABLE 5
PLGA particles - effect water phase pH

| pH | Loading (%)^{a} | Size (μm) |
|---|---|---|
| 5.0 | 2.8 | 10.5 |
| 6.8 | 4.2 | 10.1 |
| 7.4 | 687 | 7.3 |
| 9.0 | No good Particles form | |

200 mg PLGA 2A, +2 mL CH₂Cl₂ + DMSO 0.5 mL,
^{a}w/w

TABLE 6
PLGA particles - blend with PEG-
PLGA(50:50, 5K, 45K, 10% PEG)

| polymer | Loading (%)^{a} | Size (μm) |
|---|---|---|
| PEG-PLGA(10%), PLGA 2A(90%) | 1.9 | 11.5 |

Polymer 200 mg, +2 mL CH₂Cl₂ + DMSO 1 mL,
^{a}w/w

TABLE 7
PLGA particles - effect of polymer and end group

| polymer | End group | Loading (%)^{a} | Size (μm) |
|---|---|---|---|
| 7E | ester | 0.9 | 14.4 |
| 7E + TEA | ester | 1.2 | 16.6 |
| 7A | acid | 1.3 | 15.1 |
| 7A + TEA | acid | 2.4 | 20.9 |

PLGA 7A(E) 180 mg, PEG (10%, 5K)-PLGA (50:50, 45K) 20 mg + 4 mL CH₂Cl₂ + 0.5 mL DMSO, water phase pH = 6.8,
^{a}w/w

TABLE 8
PLGA particles - effect of TEA

| TEA | Loading (%)^{a} | Size (μm) |
|---|---|---|
| 0 | 11.3 | 27.7 |
| 100 | 13.9 | 29.8 |
| 200 | 16.6 | 31.1 |
| 300 | 7.5 | 30.7 |

PLGA 4A 180 mg, PEG-PLGA50:50 (5K, 45K) 20 mg, 1 mL CH₂Cl₂ + DMSO 0.5 mL, water phase pH = 6.8,
^{a}w/w

TABLE 9
PLA particles - effect of TEA

| TEA | Loading (%)^{a} | Size (μm) |
|---|---|---|
| 0 | 11.5 | 27.7 |
| 200 | 15.9 | 31.1 |

PLA 4.5E 180 mg, PEG-PLGA50:50 (5K, 45K) 20 mg + 2 mL CH₂Cl₂ + DMSO 1 mL, water phase pH = 6.8,
^{a}w/w

TABLE 10
PLGA particles - effect of 2A and 7A ratio

| polymer | Loading (%)^{a} | Size (μm) |
|---|---|---|
| 7A | 6.7 | 13.9 |
| 7E | 4.5 | 13.3 |

PLGA 7A (or 7E) 160 mg, PLGA 2A 38 mg, PEG-PLGA50:50 (5K, 45K) 2 mg + 2 mL CH₂Cl₂ + DMSO 1 mL, water phase pH = 6.8,
^{a}w/w

TABLE 11
PLGA particles - effect of 2A and 7A ratio

| polymer | Loading (%)^{a} | Size (μm) |
|---|---|---|
| 7A | 3.3 | 17.5 |
| 7E | 2.4 | 17.9 |

PLGA 7A (or 7E) 180 mg, PLGA 2A 18 mg, PEG-PLGA 50:50 (5K, 45K) 2 mg + 2 mL CH₂Cl₂ + DMSO 1 mL, water phase pH = 6.8,
^{a}w/w

TABLE 12
PLGA particles - effect of 2A and 7A ratio

| polymer | Loading (%)^{a} | Size (μm) |
|---|---|---|
| 7A | 1.9 | 11.5 |
| 7E | 1.2 | 12.8 |

PLGA 7A (or 7E) 140 mg, PLGA 2A 58 mg, PEG-PLGA50:50 (5K, 45K) 2 mg, +2 mL CH₂Cl₂ + DMSO 1 mL water phase pH = 6.8,
^{a}w/w

TABLE 13
PLGA particles -PEG-PLGA50:50(5K, 45K) particles

| polymer | Loading (%)^{a} | Size (μm) |
|---|---|---|
| PEG-PLGA | 2.9 | 22.3 |

Polymer 200 mg, +2 mL CH₂Cl₂ + DMSO 1 mL, water phase pH = 6.8,
^{a}w/w

TABLE 14
PLGA particles - effect of TEA

| TEA | Loading (%)^{a} | Size (μm) |
|---|---|---|
| 100 | 15.8 | 33.7 |
| 150 | 18.3 | 34.1 |
| 200 | 17.6 | 32.9 |

PLGA 7A 180 mg, PLGA 2A 20 mg + PEG-PLGA50:50 (5K, 45K) 2 mg, +1 mL CH₂Cl₂ + DMSO 0.5 mL, water phase pH = 6.8,
^{a}w/w

These results show that polymers with acid end groups yield higher loading. The results also show that any of (i) lower molecular weight polymers (Table 2), (ii) higher polymer concentration (Table 3), and (iii) neutral pH in water phase, gave rise to higher loading.

### Example 2. Mouse Model of Choroidal Neovascularization (CNV)

### Materials and Methods

### Mouse Model of CNV

Female C57BL/6 mice (Charles River Labs, Frederick, Md.), 4-6 weeks of age, were sedated with ketamine/xylazine (Henry Schein Animal Health, Dublin, Ohio) and using pulled glass pipettes and a microinjector (Harvard Apparatus, Holliston, Mass.) were given a 1 μl intravitreous injection of 29.4 μg PLGA-ACF MPs containing 2 μg of ACF in one eye and 29.4 μg of empty PLGA MPs in the other eye. The microparticle size was 7.3±1.8 μm and the ACF loading was 6.8% w/w. At 2, 4, 8 or 12 weeks after MP injection, Bruch's membrane was ruptured at 3 locations in each. After 1 week, mice were euthanized and choroidal flat mounts were stained with FITC-Griffonia Simplicifolia lectin which selectively stains vascular cells.

Choroidal NV was induced by laser photocoagulation-induced rupture of Bruch's membrane. Briefly, 5-6-week-old female C57BL/6 mice were anesthetized with ketamine hydrochloride (100 mg/kg body weight) and pupils were dilated. Laser photocoagulation (75 μm spot size, 0.1 sec duration, 120 mW) was performed in the 9, 12, and 3 o'clock positions of the posterior pole of each eye with the slit lamp delivery system of an OcuLight GL diode laser (Index, Mountain View, Calif.) and a handheld cover slip as a contact lens to view the retina. Production of a bubble at the time of laser, which indicates rupture of Bruch's membrane, is an important factor in obtaining choroidal NV; therefore, only burns in which a bubble was produced were included in the study. Immediately after laser-induced rupture of Bruch's membrane, mice were randomized to various treatment groups including intravitreous injections of 5 μL of phosphate-buffered saline (PBS) or HA containing 10 μg acriflavine containing microparticles, Intravitreous injections were done under a dissecting microscope with a Harvard Pump Microinjection System and pulled glass micropipettes.

After 14 days, the mice were perfused with 1 ml of PBS containing 50 mg/ml of fluorescein-labeled dextran (2×106 Daltons average molecular weight; Sigma-Aldrich, St. Louis, Mo.) and choroidal flat mounts were examined by fluorescence microscopy. Images were captured with a Nikon Digital Still Camera DXM1200 (Nikon Instruments Inc., New York, N.Y.). Image analysis software (Image-Pro Plus; Media Cybernetics, Silver Spring, Md.) was used to measure the total area of choroidal NV at each rupture site with the investigator masked with respect to treatment group.

### Results

### Intravitreous Injection of PLGA-ACF MPs Suppresses Choroidal NV in Mice for at Least 8 Weeks

C57BL/6 mice were given an intravitreous injection of 2 μg of PLGA-ACF MPs in one eye and 2 μg of empty MPs in the other eye. At 2, 4, 8, and 12 weeks after injection, Bruch's membrane was ruptured by laser photocoagulation at 3 locations in each eye. After 1 week, mice were euthanized and choroidal flat mounts were stained with FITC-Griffonia Simplicifolia lectin which selectively stains vascular cells. The area of choroidal NV at Bruch's membrane rupture sites was measured with fluorescence microscopy and image analysis.

Suppression of CNV at Bruch's membrane rupture sites for 8 weeks after intravitreous injection of PLGA-ACF MPs was observed. Compared to empty MP injected eyes, areas of choroidal NV appeared smaller in eyes injected with PLGA-ACF MPs. When Bruch's membrane was ruptured at 2, 4, or 8 weeks after injection, mean (±SEM) area of choroidal NV was significantly less in PLGA-ACF MP-injected eyes compared with fellow eye controls, but when Bruch's membrane was ruptured 12 weeks after injection, there was no significant difference (FIG. 2D).

### Example 3. Rat Model of CNV

### Materials and Methods

### Rat Model of CNV and Histology

Male Norway Brown rats (Charles River Labs, Frederick, Md.), 7-8 weeks of age, were sedated with ketamine/xylazine and given a 3 μl suprachoroidal injection of 147 μg of PLGA-ACF MPs containing 10 μg acriflavine in one eye and 147 μg of empty PLGA MPs. A 27-gauge needle was used to partially penetrate the sclera at an oblique angle 2 mm posterior to the limbus. A blunt 33-gauge needle of a Hamilton syringe was inserted into the scleral opening and advanced to enter the suprachoroidal space. The plunger was slowly depressed and after 15 seconds pressure was applied to the opening with a cotton tip applicator as the needle was withdrawn. At 2, 4, 8, 12, or 16 weeks after injection, rats had laser-induced rupture of Bruch's membrane in 4 locations in each eye (100-μm spot size, 100-ms duration, 150-mW power). Fourteen days later, rats were euthanized, eyes were removed, retinas were dissected out, and eyecups were fixed and stained with 1:500 Alexa-594 labeled-GSA (Vector Laboratories, Burlingame, Calif.). The area of choroidal NV at Bruch's membrane rupture sites was measured by image analysis with the investigator masked with respect to treatment group. The mean of the 4 values in each eye was used as a single experimental value.

Localization of PLGA-ACF MPs was assessed 2 weeks after suprachoroidal injection of 3μl containing 88 μg of PLGA-ACF MPs (6 μg ACF) 2 mm posterior to the limbus at superior pole of each eye. Eyes were removed and fixed in 4% formaldehyde for 2 hours. After cryopreservation with a sucrose gradient, the eyes were embedded in OCT compound (Sakura Finetek, Torrence, Calif.) and frozen. Ten μm sections were cut through the injection site and fluorescence was imaged using and Axioskop 2 microscope (Zeiss, Oberkochen, Germany).

### Results

### Suprachoroidal Injection of PLGA ACF MPs Suppresses Choroidal NV in Rats for at Least 16 Weeks

Since MPs injected into the vitreous cavity have the potential to migrate throughout the eye, injection of MPs into the suprachoroidal space, where they are sequestered away from the retina and anterior structures of the eye (FIG. 2A), could have advantages. Due to the small size of the eye in mice, suprachoroidal injections were performed in rats. Two weeks after suprachoroidal injection of PLGA-ACF MPs containing 6 μg ACF in Brown Norway rats, light microscopy of an ocular section showed green fluorescence of ACF in the sclera, choroid, and retina of the entire quadrant of the eye where the injection was done. High magnification showed MPs in the choroid and bright fluorescence from ACF that had entered the retina. Bruch's membrane was ruptured 2, 4, 8, 12, or 16 weeks after suprachoroidal injection of 6 μg ACF in PLGA-ACF MPs in one eye and empty PLGA MPs in the other eye and 2 weeks after Bruch's membrane rupture, choroidal NV lesions appeared much smaller in ACF-PLGA MP-injected eyes.

Specifically, two weeks after injection, a frozen ocular section showed ACF fluorescence in the retina and choroid on the side of the eye that had been injected. A high magnification fluorescence microscopy image showed PLGA-ACF MPs in the choroid, bright fluorescence from ACF that had entered the retina, and less bright fluorescence from ACF that had entered the sclera. Two weeks after rupture of Bruch's membrane, choroidal flat mounts were stained with FITC-Griffonia Simplicifolia lectin. At each time point, the area of CNV at Bruch's membrane rupture sites appeared smaller in eyes that had been injected with PLGA-ACF MPs.

Suppression of CNV at Bruch's membrane rupture sites for 16 weeks after suprachoroidal injection of PLGA-ACF MPs was observed. The mean (±SEM) area of choroidal NV was significantly less in ACF-PLGA MP-injected eyes compared with contralateral controls (FIG. 3).

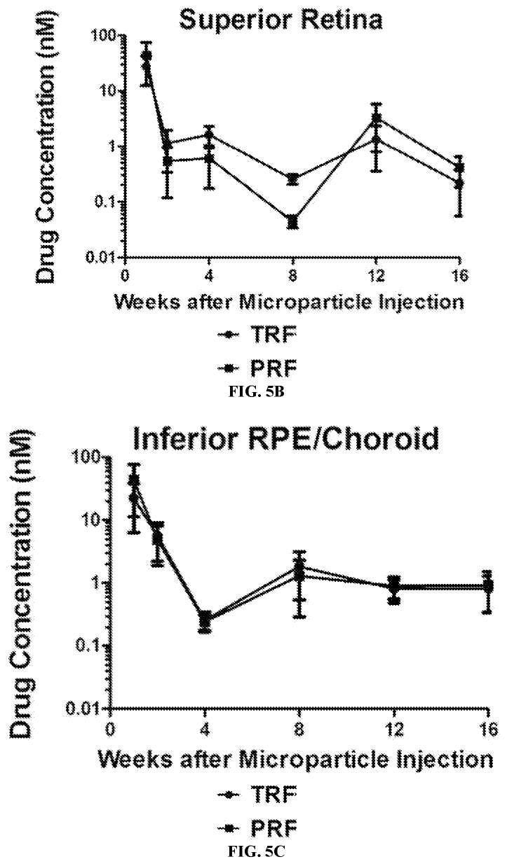

Injection of these PLGA/PVA-ACF microparticles into the SCS of rats led to a significant reduction in choroidal neovascularization for up to 16 weeks (FIG. 3). In support of the sustained therapeutic effect, sustained levels of acriflavine (mixture of trypaflavine (TRF) and proflavine (PRF)) in the retinal pigment epithelium (RPE)/choroid and the retina both superior (near the injection site) and inferior (opposite the injection site) was observed (FIGS. 5A-5D).

### Example 4. Safety Study in Rabbits

### Materials and Methods

### Rabbit Safety Study

Intraocular pressure (IOP) measurements. Dutch belted rabbit IOP was measured under gentle restraint without topical anesthesia using a tonometer (Icare TONOVET, Vantaa, Finland). IOP was measured 3-4 times per day for 7 days to establish the baseline. IOP readings are reported as the change in IOP from the baseline value for each eye.

Intraocular Injections. Rabbits were anesthetized with ketamine/xylazine, and the conjunctiva was cleaned with 5% povidone-iodine. For intravitreal injection, a 30G needle was inserted through the pars plana. For suprachoroidal injection, a 30G Hamilton Neuro Syringe with an adjustable protective needle sleeve was used and inserted above the pars plana. For both intravitreal and suprachoroidal injections, 50 μL of sodium hyaluronate solution containing either drug loaded (567 μg microparticles, 38 μg ACF) or blank microparticles (10 mg) was injected using a sterile syringe.

Fundus photography. Rabbits were anesthetized with ketamine/xylazine, and the pupils were dilated with 2.5% phenylephrine. After the application of Gonioscopic prism solution (Alcon Labs, Fort Worth, Tex.), a macula lens (Haag-streit AG, Koeniz, Switzerland) was placed above the cornea and fundus photographs were taken with a Zeiss OPMI VISU 210 Ceiling Mounted Operating Microscope with S8 Control.

Electroretinography (ERG) Measurement. Rabbits were dark adapted overnight, anesthetized with ketamine/xylazine, and placed on a heating pad set to 39° C. The pupils were dilated with 2.5% phenylephrine. Gonioscopic prism solution (Alcon Labs, Fort Worth, Tex.) was applied followed by placing platinum electrodes over both corneas. The reference electrode was attached to the scalp between the eyes and a ground electrode was inserted into the back. A ganzfeld bowl illuminator was placed above both eyes, and scotopic ERG responses were recorded (Espion ERG Diagnosys, Diagnosys, Littleton, Mass.). Recordings for both eyes were made simultaneously with the electrical impedance balanced. Four intensities (0.63, 0.4, 10, 25 (cd−s/m²)) of lights were tested.

Histology. Rabbit eyes were enucleated and immediately placed in Davidson's fixative (33% ethanol, 11.1% acetic acid, 22% neutral buffered formalin). The lens was removed from the fixed tissue before dehydrating and sending to the Johns Hopkins Reference Histology Laboratory for paraffin embedding, sectioning (5 μm thick), and H&E staining.

### Results

### Reduced Rlectroretinogram (ERG) Amplitudes after Intravitreous, but not Suprachoroidal Injections of PLGA ACF MPs in Rabbits

Safety studies were done in Dutch Belted rabbits. Mean ERG a- and b-wave amplitudes were significantly reduced at some stimulus intensities in eyes given an intravitreous (IVT) injection of 38 μg of ACF in PLGA-ACF MPs compared with eyes given an SCS injection of 38 μg of ACF in PLGA-ACF MPs into the suprachoroidal space or untreated eyes.

Change in amplitude (μV) of scotopic a-wave and scotopic b-wave at different flash intensities (cd−s/m²) of treated eyes were compared to untreated eyes of rabbits with: no injection; SCS-injected PLGA MPs, or SCS-injected PLGA-ACF MPs.

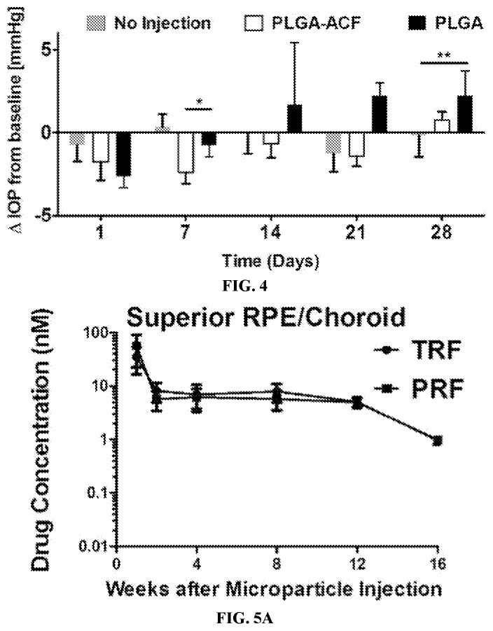

FIG. 4 is a graph showing the mean (±SEM) changes from baseline intraocular pressure (IOP) (ΔIOP from baseline [mmHg]) over time (days).

There were no differences in mean ERG a- and b-wave amplitudes in eyes given a suprachoroidal injection of either PLGA-ACF MPs or empty PLGA MPs, which were both similar to amplitudes in untreated eyes. Because of the reduced ERG function after intravitreous injection of PLGA-ACF MPs, all additional safety studies were done after suprachoroidal injection of PLGA-ACF MPs. Fundus photographs showed normal appearing peripheral retina and optic nerve in eyes given a suprachoroidal injection of PLGA-ACF MPs similar to those in untreated eyes or those given a suprachoroidal injection of empty PLGA MPs. Specifically, fundus photographs showed normal appearing peripheral retina and optic nerve 28 days after SC injection 50 μl containing 10 mg of PLGA-ACF MPs (38 μg of ACF) or 10 mg of empty PLGA MPs, indistinguishable from eyes that had no injection.

Suprachoroidal space injection (SCS), but not intravitreous injection (IVT), of PLGA-ACF MPs in rabbits is safe. Dutch belted rabbits were given a suprachoroidal (SC) or intravitreal injection of 50 μl containing 10 mg of PLGA-ACF MPs (38 μg of ACF), 10 mg of empty PLGA MPs, or no injection (n=3-4). Scotopic electroretinograms 28 days after injection showed that compared to eyes with no injection, there was no significant difference in mean (±SEM) a-wave or b-wave amplitude (μV) in eyes given a SC injection of PLGA-ACF at different flash intensities (cd−s/m²), but there was a significant reduction in mean a-wave amplitude at the highest stimulus intensity and in b-wave amplitude at the highest and lowest stimulus intensity in eyes given a an IVT injection of PLGA-ACF (*p<0.05; **p<0.01 by ANOVA with Bonferroni correction for multiple comparisons). Compared with eyes that received no injection, there was no significant reduction in a-wave or b-wave amplitudes (μV), at different flash intensities (cd−s/m²), 28 days after SC injection 50 μl containing 10 mg of PLGA-ACF MPs (38 μg of ACF) or 10 mg of empty PLGA MPs (n=3-4).

There was very little change in mean intraocular pressure (IOP) in eyes given a suprachoroidal injection of empty PLGA or PLGA-ACF MP at 1, 7, 14, 21, and 28 days after baseline, but at 7 days after injection there was a small but significant reduction in mean IOP in eyes injected with PLGA-ACF MP compared to animals injected with empty PLGA MPs, and at day 28 there was a small but significant increase in mean IOP in the empty PLGA MPs group (FIG. 4). Ocular sections showed normal retina and choroid in eyes injected with empty PLGA MPs or PLGA-ACF MPs. Specifically, changes from baseline IOP were small and not clinically significant through 28 days after SC injections of PLGA-ACF MPs or PLGA MPs, but there were some statistically significant differences between groups. At day 7, there was a significant reduction in the PLGA-ACF group compared with the others (*p<0.05 by ANOVA with Bonferroni correction for multiple comparisons) and at day 28, there was a significant increase in the PLGA group compared with no injection (**p<0.01).

Histopathology of the retina was normal 28 days after SC injection of PLGA-ACF or PLGA MPs (n=3).

### Example 5. Pharmacokinetics Study in Rats

### Materials and Methods

### Pharmacokinetics in Rats

Male Norway Brown rats, 7-8 weeks of age, received a suprachoroidal injection of 5 μl containing 147 μg of PLGA-ACF MPs (10 ACF) 2 mm posterior to the limbus at superior pole of each eye. The injection site was marked in each eye. Six rats were euthanized at each of the following time points after injection: 1 and 2 weeks, and 1, 2, 3, and 4 months. After removal of the anterior segment and lens of each eye, the retina and RPE/choroid/sclera were dissected and cut in half along the horizontal meridian through the optic nerve head and frozen separately. Samples were analyzed for acriflavine and proflavine by liquid chromatography with tandem mass spectrometry (LC-MS/MS). Tissue samples were homogenized in 200 μl of methanol and homogenized with Next Advance Bullet Blender. The analytes were extracted from 25 μl of homogenized tissue with 250 μl of acetonitrile containing internal standard (5 ng/ml of acridine orange). Samples were centrifuged and the top layer was transferred to an auto sampler vial for LC/MS/MS analysis.

Chromatographic separation was achieved with a Phenomenex Luna CN analytical column (3×50 mm, 3 μm) with a gradient. Mobile phase A was water containing 0.1% formic acid and mobile phase B was acetonitrile containing 0.1% formic acid. The gradient started with mobile phase B held at 50% for 1 minute and increased to 100% over 0.5 minutes; 100% mobile phase B was held for 1.5 minutes and then returned back to 50% mobile phase B and allowed to equilibrate for 1 minute. Total run time was 4 minutes with a flow rate of 0.6 ml/minute. The column effluent was monitored using a Sciex 4500 triple quadrupole mass spectrometer with electrospray ionization operating in positive mode. The mass spectrometer was programmed to monitor the following Multiple Reaction Monitoring transitions: 224.0>182.1 for acriflavine, 209.9>166.1 for proflavine and 266.1>234.1 for the internal standard acridine orange. The calibration curve was computed using area ratio peak of the analyte to the internal standard by using a quadratic equation with a 1/x2 weighting function over the range of 0.5-1,000 ng/ml.

### Results

### Tissue Levels of the Components of ACF in Ocular Tissues after Suprachoroidal Injection of PLGA-ACF MPs

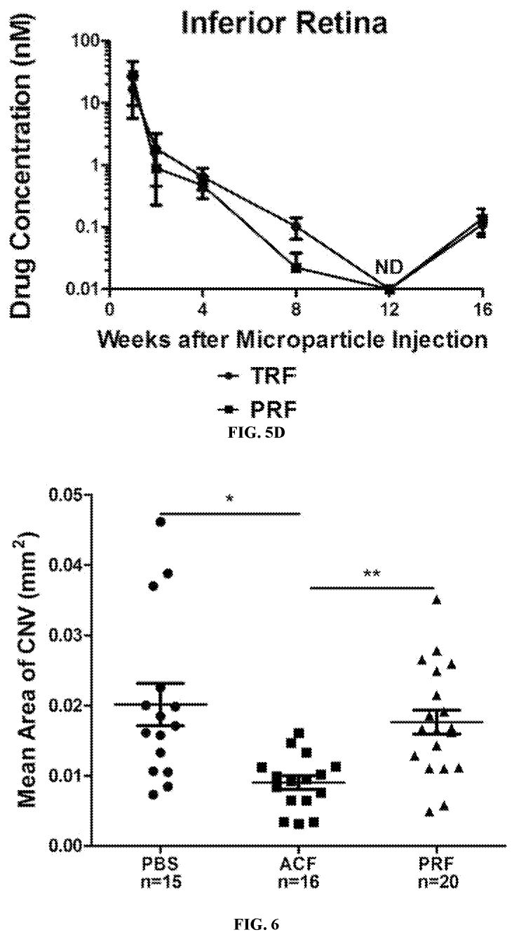

Acriflavine is a mixture of trypaflavine (TRF) and proflavine (PRF). PRF is the precursor for TRF and is difficult to separate from TRP, and therefore pure TRF is not available. In contrast to ACF, PRF does not suppress choroidal NV, showing that TRF is the active component of ACF (FIG. 6). An LC-MS assay was developed to measure TRF and PRF in ocular tissues after suprachoroidal injection of PLGA-ACF MPs containing 10 μg ACF in Brown Norway rats. The suprachoroidal injections were done at the superior pole of the eye and to determine if levels were constant throughout the entire eye, retinas and eye cups (RPE/choroid consisting of the RPE, choroid, and sclera) were dissected and divided into superior and inferior halves. There was very little difference in TRF and PRF levels in either tissue at any time point; both were above 10 nM at 1 week after injection in the superior and inferior half of retina and RPE/choroid. There was ≥10-fold drop in levels between 1 and 2 weeks after which levels were fairly stable through 16 weeks (FIGS. 5A-5D). Steady-state levels were approximately 2-8 nM and 0.5-1 nM in superior and inferior RPE/choroid, respectively, and 0.5-1 nM and 0.1 nM in superior and inferior retina, respectively.

Specifically, Brown Norway rats were given a suprachoroidal injection of 5 μl containing 147 μg of PLGA-ACF MPs (10 μg of ACF) in each eye. At 1, 2, 4, 8, 12, and 16 weeks after injection, three rats were euthanized, eyes removed (n=6 for each time point) and the retina (superior retina, FIG. 5B; inferior retina, FIG. 5D) and RPE/choroid (superior RPE/choroid, FIG. 5A; inferior RPE/Choroid, FIG. 5C) was dissected and cut into superior (side of the eye that received the SC injection) and inferior halves. The level of each component of ACF, Trypaflavine (TRF) and Proflavine (PRF), were measured by LC-MS.

### Example 6. ACF Suppression of CNV is Due to trypaflavine but not proflavine

### Materials and Methods

Mice received a 1 μl intravitreous injection of 50 ng ACF, 50 ng of proflavine (PRF), or PBS immediately after rupture of Bruch's membrane. Seven days after rupture of Bruch's membrane, mice were euthanized, eyecups were fixed and stained with FITC-labeled Griffonia Simplicifolia lectin (GSA, Vector Laboratories, Burlingame, Calif.), and flat mounted. The area of choroidal NV at each Bruch's membrane rupture site was measured by image analysis by an observer masked with respect to treatment group. The area of choroidal NV at the 3 rupture sites in one eye was averaged to give one experimental value for each animal.

C57B¹/₆ mice were given a 1 μl intraocular injection of 50 ng ACF, 50 ng PRF, or PBS in each eye immediately following laser rupture of Bruch's membrane at 3 sites. One week after rupture of Bruch's membrane, choroidal flat mounts were stained with FITC-Griffonia simplicifolia lectin.

### Results

In eyes treated with ACF the area of CNV at Bruch's membrane rupture sites appeared smaller compared to eyes that had been injected with PRF, which appeared similar to those injected with PBS only. Quantification confirmed a statistically significant reduction of CNV size in eyes treated with ACF compared to those in eyes treated with either PRF or PBS. The mean value for eyes treated with PRF was almost identical to that of eyes treated with PBS (FIG. 6).

### Example 7. SCS Injection of Microparticle Formulation Containing doxorubicin is Significantly More Advantageous than Their Intravitreal Injection

### Materials and Methods

Inflammatory reactions to particulates injected into the eye can be reduced with compositions that include a hydrophilic coating that is covalently or non-covalently associated with the particle core. This coating provides reduced inflammation or intraocular pressure (IOP) after administration of the drug delivery system to the eye, as compared to an uncoated particle.

Any inflammatory reaction to a given sustained delivery composition upon injection into, for example, the vitreous of the eye, can be reduced by injection of the same composition into the space between the sclera and choroid that traverses the circumference of the posterior segment (suprachoroidal space, SCS). An additional advantage to injection of a sustained release composition into the SCS is that, in contrast to injection into the vitreous, there is no concern about the composition entering the visual axis.

Two formulations were prepare and tested. The first formulation containing the small molecule drug acriflavine (ACF) was composed of a poly(lactic-co-glycolic acid) (PLGA) core and a polyvinyl alcohol (PVA) coating. The same volume and concentration of particles was injected either IVT or SCS in healthy Dutch belted rabbit eyes. This is the same PLGA/PVA formulation of ACF used in the earlier examples with data shown in FIGS. 1A, 2, 3, 4, or 5.

The second formulation contained microparticles doxorubicin (DXR) covalently attached to poly(sebacic acid)-(polyethylene glycol)₃ grafted copolymers (DXR-PSA-PEG₃). The microparticles were prepared as described in Iwase et al., J Control Release, 172(3):625-633 (2013).

### Results

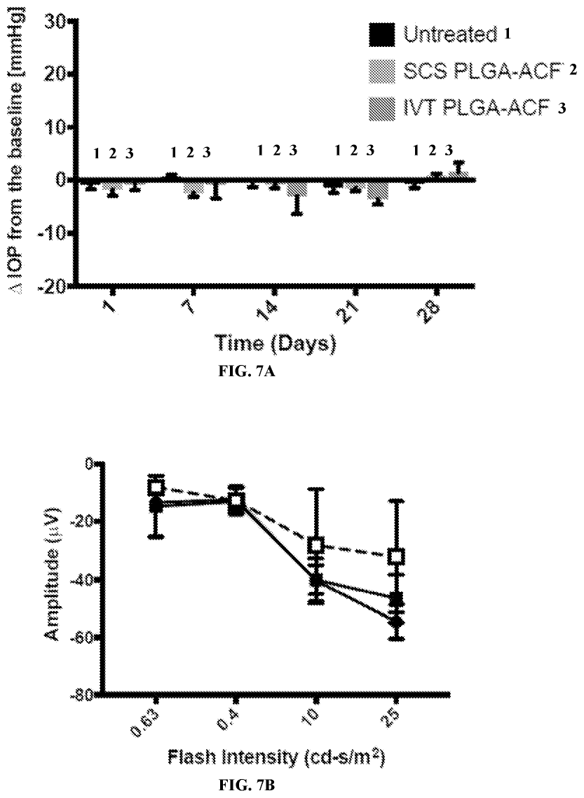

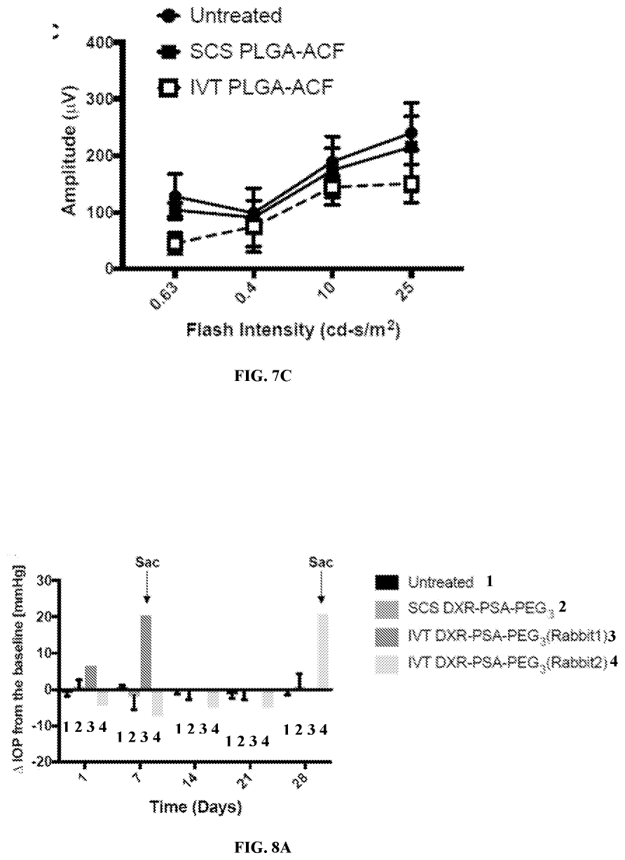

Biodegradable microparticle formulations that showed safety concerns upon intravitreal (IVT) injection were well-tolerated with injection into the SCS. While there was no change in IOP over the first 28 days (FIG. 7A), a shift in the a-wave (FIG. 7B) and b-waves (FIG. 7C) measured by electroretinography (ERG) was observed with IVT injection only. It should be noted that FIG. 7A has some data overlapping with FIG. 4, in terms of the SCS PLGA-ACF particles. While FIG. 7 compares their effect SCS PLGA-ACF particles to IVT PLGA-ACF particles, FIG. 4 compares SCS PLGA-ACF to SCS blank PLGA particles.

In support of alteration of retinal function, fundus imaging further revealed signs of retinal necrosis only in the rabbits receiving IVT particle injections. The retinas of animals receiving particle injections into the SCS looked similar to those of untreated rabbits.

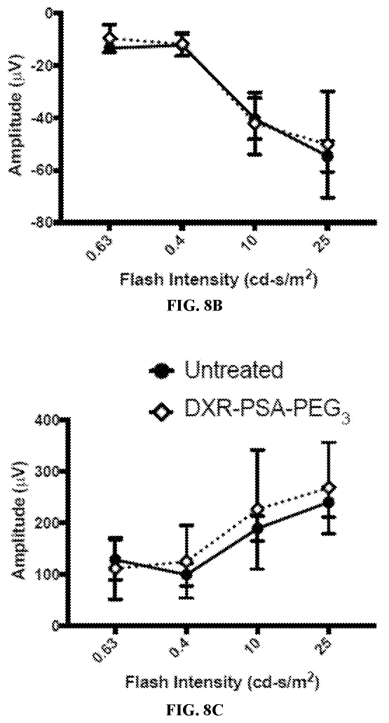

Microparticles containing doxorubicin (DXR) covalently attached to poly(sebacic acid)-(polyethylene glycol)₃ grafted copolymers (DXR-PSA-PEG₃) were also safe when administered to the SCS. Rapid release of the drug attached to low molecular weight polymer species caused retinal toxicity and inflammation with IVT injection that was not observed with SCS injection. As shown in FIG. 8A, rabbits receiving IVT injection of DXR-PSA-PEG₃ microparticles showed elevated IOP that required sacrificing the rabbit (Rabbit 1 on day 7 and Rabbit 2 on day 28). These rabbits further had signs of ocular cloudiness and/or hyperemia in the eye with elevated IOP that received the IVT injection. In contrast, the rabbits receiving the SCS microparticle injection had no signs of IOP elevation (FIG. 8A) or gross ocular inflammation. Further, the ERG of rabbits receiving the SCS injection was similar to untreated eyes (FIGS. 8B and 8C), whereas ERG could not be measured for the rabbits receiving IVT injection of DXR-PSA-PEG₃ microparticles because they had to be sacrificed. Additionally, there were no signs of retinal toxicity with fundus imaging after SCS injection, whereas hazy, mottled necrotic areas of the retina were observed 1 week after IVT injection.

Therefore, ERG could only be measured on animals receiving SCS injection, and the a-wave (FIG. 8B) and b-wave (FIG. 8C) were similar to those of the untreated rabbit eyes. Similarly, fundus photography revealed no evident retinal toxicity with SCS injection, whereas IVT injection led to retinal necrosis.

These methods achieve injection of sustained-release microparticles into the SCS to facilitate prolonged therapeutic effect with minimal inflammation, retinal toxicity, and effect on vision.

### Example 8. Role of acriflavine, daunorubicin, or doxorubicin on Oxygen-Induced Ischemic Retinopathy in Mice

### Materials and Methods

Mouse model of laser-induced choroidal NV Choroidal NV was generated as described by Tobe et al., Am J Pathol 153:1641-1646 (1998). Briefly, 6-week-old C57BL/6 mice had rupture of Bruch's membrane in 3 locations in each eye by laser photocoagulation and were treated with acriflavine or vehicle by various modes of administration: intraperitoneal injections, intravitreous injections, or topical administration. Seven days after rupture of Bruch's membrane, mice were euthanized, eyecups were stained with FITC-labeled GSA (Vector Laboratories, Burlingame, Calif.), and flat mounted. The area of choroidal NV at each Bruch's membrane rupture site was measured by image analysis by an observer masked with respect to treatment group. The area of choroidal NV at the three rupture sites in one eye was averaged to give one experimental value.

### Results

Results for the effect of acriflavine on the CNV and ROP models are shown in FIGS. 2B and 2C.

### Example 9. Trocar/Cannula Technique for SCS Injection

### Materials and Methods

Suprachoroidal injections are being performed in clinical trials using microneedles and it is widely held in the field that using microneedles is the only way to perform suprachoroidal injections in patients. A technique using a 27-gauge valved trocar/cannula that is widely available was developed. The eye was anesthetized using proparacaine drops and subconjunctival injection of 2% lidocaine, and then a drop of 5% betadine was applied. The 27-gauge trocar has a flat, thin blade that must be positioned with the flat portion of the blade parallel to the surface of the eye (it cannot be positioned in any other orientation, e.g. with the flat portion of the blade perpendicular to the surface of the eye). Four mm posterior to the limbus in any quadrant of the eye, the flat portion of the blade was touched to the surface of the conjunctiva with the tip of the blade directed parallel to the limbus. With the blade lying flat on the conjunctiva, it was gradually advanced with the tip of the blade oriented just slightly downward so that the blade penetrates the conjunctiva and then the sclera at a very oblique angle. The trocar/cannula was advanced slowly until a portion of the cannula was within the eye and the remainder with the hub is lying on its side on the surface of the eye. The trocar was slowly removed by holding the cannula adjacent to the hub with a forceps while slowly removing the trocar. The tip of the cannula was then sitting in the suprachoroidal space. A primed 30-gauge needle attached to a syringe containing 50 μl of fluid containing microparticles or another therapeutic was inserted through the valves into the cannula and the contents are slowly injected into the suprachoroidal space. As the contents were injected, the intraocular pressure increased limiting the volume that could be injected; 50 μl was a safe limit for injection into a human eye, because with that volume the increase in intraocular pressure is not sufficient to close the retinal circulation, however it would be prudent to examine the retinal circulation by indirect ophthalmoscopy after injection.

A major advantage of the cannula system is that it can remain in the suprachoroidal space for a prolonged period of time, allowing the intraocular pressure to return to normal making a second injection of 50 μl possible. Alternatively, after the first injection, a 30 gauge needle can be inserted into the anterior chamber and 100-200 μl of aqueous humor can be withdrawn. A second suprachoroidal injection can then be given with volume even larger than the first (up to 150 μl if 200 μl of aqueous was removed). Another alternative is to remove 100-200 μl of aqueous immediately after the cannula is inserted but before suprachoroidal injection. This allows a single suprachoroidal injection of up to 200 μl.

Thirty seconds after the final suprachoroidal injection, a cotton tip was held over the cannula entry site, the cannula was slowly withdrawn, and the cotton tip was held in place for 30 seconds.

### Results

Injections were performed on rabbit and pig eyes. Injection into rabbits' eye showed successful suprachoroidal injection with a 27-gauge trocar/cannula in a rabbit. An ocular section through a pig eye after suprachoroidal injection of 50 μl of India Ink with a 27-gauge trocar/cannula showed the ink is seen throughout the choroid from one side of the eye where the injection was done to the opposite side of the eye. The ink did not extend into the subretinal space or the retina.

The results in the Examples show that the highly water soluble ACF was loaded into PLGA microparticles (PLGA-ACF MPs) that released ACF in vitro for up to 60 days. Intravitreous injection of PLGA-ACF MPs in mice suppressed choroidal NV for at least 8 weeks and suprachoroidal injection of PLGA-ACF in rats suppressed choroidal NV for at least 16 weeks. Intravitreous, but not suprachoroidal injection, of PLGA-ACF MPs containing 38 μg of ACF in rabbits resulted in modest reduction of electroretinogram function. Over the span of 28 days after suprachoroidal injection of PLGA-ACF MP, rabbits had normal appearing retinas on fundus photographs, normal electroretinogram scotopic a- and b-wave amplitudes, no increase in intraocular pressure, and normal retinal histology. The active components of ACF, trypaflavine, had steady-state levels in the low nM range in RPE/choroid, and greater than in retina, for at least 16 weeks with a gradient from the side of the eye where the injection was done to the opposite side.

In these examples, ACF was incorporated into PLGA MPs and it was found that intravitreous injection of PLGA-ACF MPs containing 2 μg ACF suppressed choroidal NV at Bruch's membrane rupture sites for at least 8 weeks. Intravitreous injection of MPs requires modifications that promote aggregation and prevent dispersion of MPs which can degrade vision. Suprachoroidal injection sequesters MPs away from the retina, but still in close proximity allowing diffusion of ACF into the retina. Suprachoroidal injection PLGA-ACF MPs containing 6 μg ACF suppressed choroidal NV at Bruch's membrane rupture sites for at least 16 weeks. Intravitreous injection, but not suprachoroidal injection, of PLGA-ACF MPs containing 38 μg of ACF in rabbits resulted in modest reduction of ERG function, showing that sustained release of ACF in the suprachoroidal space provides an added layer of safety.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A population of polymeric particles for suprachoroidal delivery and controlled release of a therapeutic, prophylactic or diagnostic agent formulated for subchoroidal administration, comprising polymeric particles comprising
a biodegradable polymeric matrix, and
a therapeutic, prophylactic or diagnostic agent which exhibits sustained controlled release from the polymeric matrix with minimal to no burst release following suprachoroidal administration,
wherein, suprachoroidal delivery of the agent in the polymeric matrix causes less toxicity in the eye relative to intravitreal delivery of the agent.

2. The population of particles of claim 1 wherein toxicity or inflammation is determined by measuring intraocular pressure and/or electroretinogram over a defined post-delivery.

3. The population of polymeric particles of claim 1, wherein the particles contain between the agent in a loading between about 0.1% and 20% of the weight of the particles.

4. The population of polymeric particles of claim 1 wherein agent is released for a period of at least one week, two weeks, one month, two months, or three months.

5. The population of polymeric particles of claim 1, comprising a therapeutic agent, wherein the therapeutic agent is selected from the group consisting of small molecules, peptides, nucleic acids, and combinations thereof.

6. The population of polymeric particles of claim 5, wherein the therapeutic agent has a water solubility between about 1 mg/ml and 500 mg/ml at room temperature and pressure.

7. The population of polymeric particles of claim 1, wherein the particles have an average diameter between about 100 nm and about 100 μm, as measured by scanning electron microscopy.

8. The population of polymeric particles of claim 1, wherein the biodegradable polymer comprises a polymer selected from the group consisting of polyesters, polyanhydrides, polyorthoesters, blends, and co-polymers thereof.

9. The population of polymeric particles of claim 8, wherein the biodegradable polymer comprises a poly(hydroxyacids), blend, or co-polymer thereof.

10. The population of polymeric particles of claim 1, wherein the biodegradable polymer has carboxylic acid or ester end groups.

11. The population of polymeric particles of claim 1, wherein the particles further comprise comprises a polymeric coating.

12. The population of polymeric particles of claim 1, comprising a therapeutically effective amount of a small molecule selected from the group consisting of an acriflavine, a doxorubicin, and combinations thereof.

13. A method of making the population of polymeric particles of claim 1, the method comprising mixing the agent with the biodegradable polymer to form a polymer-agent mix, then processing the polymer-agent mix in a method selected from the group consisting of solvent evaporation, single emulsion solvent evaporation, nanoprecipitation, microfluidics, solvent extraction, phase inversion, and spray drying to form microparticles.

14. The method of claim 13, wherein the biodegradable polymer has a molecular weight between about 5 kDa and about 200 kDa, or between about 5 kDa and about 120 kDa, has carboxylic or ester end groups, and/or the polymer is at a concentration between about 10 mg/ml and 400 about mg/ml.

15. The method of claim 13, wherein the pH of a water phase in the single emulsion solvent evaporation method is between about 5.0 and about 9.0, such as 5.0, 6.8, 7.4, or 9.0.

16. A method for treating a subject with an ocular disease or disorder, the method comprising
administering to a suprachoroidal space (SCS) of the subject the population of polymeric particles of claim 1.

17. The method of claim 16, wherein the subject has ocular neovascularization.
